(19) United States
(12) Patent Application Publication
(10) Pub. No.: US 2025/0152716 A1
(43) Pub. Date: May 15, 2025

(54) IMPROVED PRIMARY HUMAN NK CELL EXPANSION AND FUNCTION BY CHIMERIC CYTOKINE RECEPTOR

(71) Applicant: The Regents of the University of California, Oakland, CA (US)

(72) Inventors: Avishai Shemesh, Oakland, CA (US); Lewis L. Lanier, San Francisco, CA (US); Kole Roybal, Oakland, CA (US)

(73) Assignee: The Regents of the University of California, Oakland, CA (US)

(21) Appl. No.: 18/838,202

(22) PCT Filed: Feb. 17, 2023
(86) PCT No.: PCT/US2023/062790

§ 371 Date: Aug. 13, 2024

Related U.S. Application Data
(60) Provisional application No. 63/311,702, filed on Feb. 18, 2022.

(51) Int. Cl. A61K 40/42; A61K 40/11; A61K 40/15; A61K 40/31; C07K 14/715

(52) U.S. Cl. CPC A61K 40/4218; A61K 40/11; A61K 40/15; A61K 40/31; A61K 40/4217; C07K 14/7155; C07K 14/7156

(57) ABSTRACT

Natural killer (NK) cells are innate lymphocytes with cancer and viral immunosurveillance capabilities. Prior studies have established that human and mouse NK cells can acquire features of adaptive immunity, demonstrating immunological memory-like properties. Memory-like NK cells have been described in response to infection with cytomegalovirus in humans and mice, representing antigen-specific memory NK cells. NK cells, designated as cytokine-induced memory-like NK cells, with adaptive immune cell features can be generated in vitro and used in clinical trials in human cancer patients. Chimeric transmembrane receptor polypeptides expressed in natural killer cells or T-cells, are provided.

### CROSS-REFERENCE TO RELATED PATENT APPLICATIONS

The present patent application is a 371 U.S. National Phase Application of PCT/US2023/062790, filed Feb. 17, 2023, which claims benefit of priority to U.S. Provisional Patent Application No. 63/311,702, filed Feb. 18, 2022, both of which are incorporated by reference for all purposes.

### SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Feb. 16, 2023, is entitled “081906-1371671-246710PC_ST26.xml”, and is 147,791 bytes in size.

### BACKGROUND OF THE INVENTION

Natural killer (NK) cells are innate lymphocytes with cancer and viral immunosurveillance capabilities (Cerwenka and Lanier, Nat Rev Immunol. 16:112-123, 2016). Prior studies have established that human and mouse NK cells can acquire features of adaptive immunity, demonstrating immunological memory-like properties (Sun et al., Nature 457:557-561, 2009). Memory-like NK cells have been described in response to infection with cytomegalovirus in humans and mice, representing antigen-specific memory NK cells (Sun et al., Nature 457:557-561, 2009; Lee et al., J Exp Med. 206:2235-2251, 2009). NK cells, designated as cytokine-induced memory-like NK cells, with adaptive immune cell features can be generated in vitro and used in clinical trials in human cancer patients (Ni et al., J Exp Med. 209:2351-2365, 2012; Cooper et al., PNAS 106:1915-1919 2009; Romee et al., Science Translational Medicine 8:357 ra123-357, 2016).

### BRIEF SUMMARY OF THE INVENTION

In some embodiments, a human natural killer cell or T-cell expressing a first and second chimeric transmembrane protein is provided. In some embodiments, the first chimeric transmembrane protein comprises a first ligand-binding ectodomain linked to one or more intracellular signaling domains from a human IL-12 receptor or IL-15 receptor; and the second chimeric transmembrane protein comprises a second ligand-binding ectodomain linked to one or more intracellular signaling domains from a human IL-12 receptor or IL-15 receptor or other interleukin receptor, wherein the first and second ligand-binding ectodomains together bind the ligand to trigger signaling by the intracellular signaling domains.

In some embodiments, intracellular signaling domains of the first chimeric transmembrane protein and the second chimeric transmembrane protein are human IL-12 receptor signaling domains. In some embodiments, at least one of the one or more intracellular signaling domains in the first chimeric transmembrane receptor comprise a signaling domain from human IL12RB1, and at least one of the one or more intracellular signaling domains in the second chimeric transmembrane receptor comprise a signaling domain from human IL12RB2. In some embodiments, the signaling domain from human IL12RB1 comprises an amino acid sequence at least 90% (e.g., 91, 92, 93, 94, 95, 96, 97, 98, 99, or 100%) identical to SEQ ID NO: 7; and the signaling domain from human IL12RB2 comprises an amino acid sequence at least 90% (e.g., 91, 92, 93, 94, 95, 96, 97, 98, 99, or 100%) identical to SEQ ID NO: 8.

In some embodiments, intracellular signaling domains of the first chimeric transmembrane protein and the second chimeric transmembrane protein are human IL-15 receptor signaling domains. In some embodiments, at least one of the one or more intracellular signaling domains in the first chimeric transmembrane receptor comprise a signaling domain from human IL2RB, and at least one of the one or more intracellular signaling domains in the second chimeric transmembrane receptor comprise a signaling domain from human IL2RG. In some embodiments, the signaling domain from human IL2RB comprises an amino acid sequence at least 90% (e.g., 91, 92, 93, 94, 95, 96, 97, 98, 99, or 100%) identical to SEQ ID NO: 9; and the signaling domain from human IL2RG comprises an amino acid sequence at least 90% (e.g., 91, 92, 93, 94, 95, 96, 97, 98, 99, or 100%) identical to SEQ ID NO: 10.

In some embodiments, the ligand is IFNγ and the first ligand-binding ectodomain comprises a human IFNγR1 IFNγ-binding domain and the second ligand-binding ectodomain comprises a human IFNγR2 IFNγ-binding domain. In some embodiments, ligand is IFNγ and the first ligand-binding ectodomain comprises a human IFNγR2 IFNγ-binding domain and the second ligand-binding ectodomain comprises a human IFNγR1 IFNγ-binding domain. In some embodiments, the human IFNγR1 IFNγ-binding domain comprises an amino acid sequence at least 90% (e.g., 91, 92, 93, 94, 95, 96, 97, 98, 99, or 100%) identical to SEQ ID NO: 1; and the human IFNγR2 IFNγ-binding domain comprises an amino acid sequence at least 90% (e.g., 91, 92, 93, 94, 95, 96, 97, 98, 99, or 100%) identical to SEQ ID NO: 2.

In some embodiments, the ligand is GM-CSF and the first ligand-binding ectodomain comprises a human CSF2RA GM-CSF binding domain and the second ligand-binding ectodomain comprises a human CSF2RB GM-CSF-binding domain. In some embodiments, the ligand is GM-CSF and the first ligand-binding ectodomain comprises a human CSF2RB GM-CSF-binding domain and the second ligand-binding ectodomain comprises a human CSF2RA GM-CSF-binding domain. In some embodiments, the human CSF2RA GM-CSF-binding domain comprises an amino acid sequence at least 90% (e.g., 91, 92, 93, 94, 95, 96, 97, 98, 99, or 100%) identical to SEQ ID NO: 3; and the human CSF2RB GM-CSF-binding domain comprises an amino acid sequence at least 90% (e.g., 91, 92, 93, 94, 95, 96, 97, 98, 99, or 100%) identical to SEQ ID NO: 4.

In some embodiments, the ligand is TGFβ and the first ligand-binding ectodomain comprises a human TGFBR1 TGFβ-binding domain and the second ligand-binding ectodomain comprises a human TGFBR2 TGFβ-binding domain. In some embodiments, the ligand is TGFβ and the first ligand-binding ectodomain comprises a human TGFBR2 TGFβ-binding domain and the second ligand-binding ectodomain comprises a human TGFBR1 TGFβ-binding domain. In some embodiments, the human TGFBR1 TGFβ-binding domain comprises an amino acid sequence at least 90% (e.g., 91, 92, 93, 94, 95, 96, 97, 98, 99, or 100%) identical to SEQ ID NO: 5; and the human TGFBR2 TGFβ-binding domain comprises an amino acid sequence at least 90% (e.g., 91, 92, 93, 94, 95, 96, 97, 98, 99, or 100%) identical to SEQ ID NO: 6.

In some embodiments, at least one of the one or more intracellular signaling domains in the first chimeric transmembrane receptor comprise a signaling domain from human IL12RB1, and at least one of the one or more intracellular signaling domains in the second chimeric transmembrane receptor comprise a signaling domain from human IL23R; or
wherein at least one of the one or more intracellular signaling domains in the first chimeric transmembrane receptor comprise a signaling domain from human IL12RB2, and at least one of the one or more intracellular signaling domains in the second chimeric transmembrane receptor comprise a signaling domain from human GP130; or
wherein at least one of the one or more intracellular signaling domains in the first chimeric transmembrane receptor comprise a signaling domain from human IL12RB2, and at least one of the one or more intracellular signaling domains in the second chimeric transmembrane receptor comprise a signaling domain from human IL27RA or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL21R or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL4R or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL7R or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL9R; or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL12RB1; or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL12RB2; or
wherein at least one of the one or more intracellular signaling domains in the first chimeric transmembrane receptor comprise a signaling domain from human IL2RB, and at least one of the one or more intracellular signaling domains in the second chimeric transmembrane receptor comprise a signaling domain from human IL12RB1; or
wherein at least one of the one or more intracellular signaling domains in the first chimeric transmembrane receptor comprise a signaling domain from human IL2RB, and at least one of the one or more intracellular signaling domains in the second chimeric transmembrane receptor comprise a signaling domain from human IL12RB2.

In some embodiments, the human natural killer cell is a primary natural killer cell or derived from an induced pluripotent stem cell.

Also provided is a first nucleic acid and a second nucleic acid, the first nucleic acid encoding a first chimeric transmembrane protein that comprises a first ligand-binding ectodomain linked to one or more intracellular signaling domains from a human IL-12 receptor or IL-15 receptor; and the second nucleic acid encoding a second chimeric transmembrane protein that comprises a second ligand-binding ectodomain linked to one or more intracellular signaling domains from a human IL-12 receptor or IL-15 receptor, wherein when the first chimeric transmembrane protein and the second chimeric transmembrane protein are expressed in a cell in the presence of the ligand, the first and second ligand-binding ectodomains together bind the ligand to trigger signaling by the intracellular signaling domains.

In some embodiments, intracellular signaling domains of the first chimeric transmembrane protein and the second chimeric transmembrane protein are human IL-12 receptor signaling domains.

In some embodiments, at least one of the one or more intracellular signaling domains in the first chimeric transmembrane receptor comprise a signaling domain from human IL12RB1, and at least one of the one or more intracellular signaling domains in the second chimeric transmembrane receptor comprise a signaling domain from human IL12RB2. In some embodiments, the signaling domain from human IL12RB1 comprises an amino acid sequence at least 90% (e.g., 91, 92, 93, 94, 95, 96, 97, 98, 99, or 100%) identical to SEQ ID NO: 7; and the signaling domain from human IL12RB2 comprises an amino acid sequence at least 90% (e.g., 91, 92, 93, 94, 95, 96, 97, 98, 99, or 100%) identical to SEQ ID NO: 8.

In some embodiments, intracellular signaling domains of the first chimeric transmembrane protein and the second chimeric transmembrane protein are human IL-15 receptor signaling domains.

In some embodiments, at least one of the one or more intracellular signaling domains in the first chimeric transmembrane receptor comprise a signaling domain from human IL2RB, and at least one of the one or more intracellular signaling domains in the second chimeric transmembrane receptor comprise a signaling domain from human IL2RG. In some embodiments, the signaling domain from human IL2RB comprises an amino acid sequence at least 90% (e.g., 91, 92, 93, 94, 95, 96, 97, 98, 99, or 100%) identical to SEQ ID NO: 9; and the signaling domain from human IL2RG comprises an amino acid sequence at least 90% (e.g., 91, 92, 93, 94, 95, 96, 97, 98, 99, or 100%) identical to SEQ ID NO: 10.

In some embodiments, the ligand is IFNγ and the first ligand-binding ectodomain comprises a human IFNγR1 IFNγ-binding domain and the second ligand-binding ectodomain comprises a human IFNγR2 IFNγ-binding domain In some embodiments, the ligand is IFNγ and the first ligand-binding ectodomain comprises a human IFNγR2 IFNγ-binding domain and the second ligand-binding ectodomain comprises a human IFNγR1 IFNγ-binding domain. In some embodiments, wherein the human IFNγR1 IFNγ-binding domain comprises an amino acid sequence at least 90% (e.g., 91, 92, 93, 94, 95, 96, 97, 98, 99, or 100%) identical to SEQ ID NO: 1 and the human IFNγR2 IFNγ-binding domain comprises an amino acid sequence at least 90% (e.g., 91, 92, 93, 94, 95, 96, 97, 98, 99, or 100%) identical to SEQ ID NO: 2.

In some embodiments, the ligand is GM-CSF and the first ligand-binding ectodomain comprises a human CSF2RA GM-CSF binding domain and the second ligand-binding ectodomain comprises a human CSF2RB GM-CSF-binding domain. In some embodiments, the ligand is GM-CSF and the first ligand-binding ectodomain comprises a human CSF2RB GM-CSF-binding domain and the second ligand-binding ectodomain comprises a human CSF2RA GM-CSF-binding domain. In some embodiments, the human CSF2RA GM-CSF-binding domain comprises an amino acid sequence at least 90% (e.g., 91, 92, 93, 94, 95, 96, 97, 98, 99, or 100%) identical to SEQ ID NO: 3; and the human CSF2RB GM-CSF-binding domain comprises an amino acid sequence at least 90% (e.g., 91, 92, 93, 94, 95, 96, 97, 98, 99, or 100%) identical to SEQ ID NO: 4.

In some embodiments, the ligand is TGFβ and the first ligand-binding ectodomain comprises a human TGFBR1 TGFβ-binding domain and the second ligand-binding ectodomain comprises a human TGFBR2 TGFβ-binding domain. In some embodiments, the ligand is TGFβ and the first ligand-binding ectodomain comprises a human TGFBR2 TGFβ-binding domain and the second ligand-binding ectodomain comprises a human TGFBR1 TGFβ-binding domain. In some embodiments, the human TGFBR1 TGFβ-binding domain comprises an amino acid sequence at least 90% (e.g., 91, 92, 93, 94, 95, 96, 97, 98, 99, or 100%) identical to SEQ ID NO: 5; and the human TGFBR2 TGFβ-binding domain comprises an amino acid sequence at least 90% (e.g., 91, 92, 93, 94, 95, 96, 97, 98, 99, or 100%) identical to SEQ ID NO: 6.

In some embodiments, at least one of the one or more intracellular signaling domains in the first chimeric transmembrane receptor comprise a signaling domain from human IL12RB1, and at least one of the one or more intracellular signaling domains in the second chimeric transmembrane receptor comprise a signaling domain from human IL23R; or
wherein at least one of the one or more intracellular signaling domains in the first chimeric transmembrane receptor comprise a signaling domain from human IL12RB2, and at least one of the one or more intracellular signaling domains in the second chimeric transmembrane receptor comprise a signaling domain from human GP130; or
wherein at least one of the one or more intracellular signaling domains in the first chimeric transmembrane receptor comprise a signaling domain from human IL12RB2, and at least one of the one or more intracellular signaling domains in the second chimeric transmembrane receptor comprise a signaling domain from human IL27RA or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL21R or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL4R or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL7R or
wherein at least one of the one or more intracellular signaling domains in the first chimeric transmembrane receptor comprise a signaling domain from human IL2RG, and at least one of the one or more intracellular signaling domains in the second chimeric transmembrane receptor comprise a signaling domain from human IL9R.

In some embodiments, the first nucleic acid and the second nucleic acid are linked together as one polynucleotide. In some embodiments, the first nucleic acid and second nucleic acid comprise a single open reading frame that encodes the first chimeric transmembrane protein linked to the second chimeric transmembrane protein via a cleavable amino acid sequence. In some embodiments, the cleavable amino acid sequence comprises one or more of a T2a peptide sequence, a P2A peptide sequence, an E2A peptide sequence, an F2A peptide sequence or a furin-cleavable sequence.

In some embodiments, the first nucleic acid and the second nucleic acid are separate polynucleotides not linked together.

Also provided is a vector comprising the one polynucleotide as described above or elsewhere herein. In some embodiments, the vector is a viral vector or a plasmid.

Also provided is a cell comprising the first nucleic acid and the second nucleic acid as descried above or comprising the vector as described above.

Also provided is a method of making a human natural killer cell or T-cell expressing a first and second chimeric transmembrane protein. In some embodiments, the first chimeric transmembrane protein comprises a first ligand-binding ectodomain linked to one or more intracellular signaling domains from a human IL-12 receptor or IL-15 receptor; and the second chimeric transmembrane protein comprises a second ligand-binding ectodomain linked to one or more intracellular signaling domains from a human IL-12 receptor or IL-15 receptor, respectively, and the first and second ligand-binding ectodomains together bind the ligand to trigger signaling by the intracellular signaling domains, the method comprising, introducing the first nucleic acid and the second nucleic acid as described herein into a human natural killer cell or T-cell under conditions to allow for expression of the first and second chimeric transmembrane protein. In some embodiments, the natural killer cell is a primary natural killer cell. In some embodiments, following the introducing, administering the natural killer cells or T-cells to a human. In some embodiments, the natural killer cells or T-cells are autologous or allogenic to the human.

Also provided is a method of stimulating natural killer cell or T-cell proliferation. In some embodiments, the method comprises contacting a ligand to natural killer cells or T-cells expressing the first and second chimeric transmembrane protein as described above or elsewhere herein, wherein the first and second ligand-binding ectodomains together bind the ligand to trigger signaling by the intracellular signaling domains and stimulates natural killer cell or T-cell proliferation. In some embodiments, the contacting is performed in vitro. In some embodiments, the contacting is performed in vivo or ex vivo. In some embodiments, the natural killer cell or T-cell produce the ligand.

### BRIEF DESCRIPTION OF THE DRAWINGS

FIG. 1A-B: Chimeric human IFNγR-human IL-12R or chimeric human IFNγR-human IL-2R (CC12R or CC2R) design and expression in human NK cell line NK92. NK92, which is responsive to IL-2, IL-15 or IL-12 signaling (require IL-2 or IL-15 or IL-12 for survival and expansion), was transduced with lentiviral particles containing the CC12R (A) or CC2R (B). CCR⁺ NK92 were cultured in the presence of IFNγ (100 ng/ml) without IL-2, IL-15, or IL-12. Flow cytometry analysis of CCR expression in live NK92 cells. INFγR1-IL12RB1 chain and IFNγR1-IL2RB chains were detected by anti-myc-tag antibody. INFγR2-IL12RB2 and IFNγR2-IL2RG chains were detected by anti-FLAG antibody. Dead cells were excluded by fixable-near infrared live/dead dye.

FIG. 2A-B: Chimeric human IFNγR-human IL-12 receptor (CC12R) mediates IFNγ-dependent cell proliferation, survival, and expansion of human NK cell line NK92. CC12R− (A), CC12R+ (B). NK92 cells (2×10⁵ cells/well) were cultured in 2 ml media in 24 well plates. On days 0, 2, and 4, 500 ul of media with or without the indicated cytokines (200 U/ml IL-2, 100 ng/ml IFNγ) were added. Cell proliferation, survival, and expansion were evaluated by flow cytometry analysis on days 1-6. Cells were pre-labeled using cell tracer violet (CTV, dye is diluted during cell division) to evaluate cell proliferation. Cells were stained each day with near-infrared live/dead dye to exclude dead cells and to evaluate the percentage of live cells. Cell expansion was evaluated daily to calculate the relative number of live cells between the culture conditions.

FIG. 3A-C: Chimeric human IFNγR-human IL12R (CCR) transduction of human primary NK cells: Purified human primary peripheral blood NK cells from four donors (S71-S74) were cultured for 3 days with or without lentiviral particles containing the CC12R constructs. Flow cytometry analysis of CC12R expression by anti-myc-tag antibody (1^{st} CC12R chain) and anti-FLAG antibody (2^{nd} CC12R chain). Live cells percentage were assessed by using a near-infrared live/dead dye to exclude dead cells. Expression was evaluated relative to a donor-matched control sample (CC12R−). FIG. 3A: Expression of CC12R after 3 days of culture. FIG. 3B: Percentage of CC12R+ cells evaluated by FLAG and myc expression. FIG. 3C: Percentage of live cells. Paired t-test, parametric, *p<0.05. Mean+/−standard deviation (S.D).

FIG. 4A-C: Chimeric human IFNγR-human IL12R receptor (CC12R) expression in human primary peripheral blood NK cells. CC12R-transduced purified human primary NK cells from four donors were cultured for 6 days with the indicated stimulation. CC12R expression was evaluated by flow cytometry analysis using anti FLAG antibody (2^{nd} CC12R chain) on CD3-CD56+ NK cells. Cell viability was assessed using a near-infrared live/dead dye. Expression was evaluated relative to donor-matched control samples (CC12R−). FIG. 4A: Cell viability. FIG. 4B: Percentage of CC12R+ NK cells evaluated by FLAG expression. FIG. 4C: Expression of the NK cell marker CD56 (Y-axis) vs. FLAG [CC12R] (X-axis) with the dependency of IL-2 or IL-12 stimulation. A and B, human IL-12^{low}=2.5 ng/ml, human IL-2^{low}=3 U/ml; C, human IL-12=2.5 ng/ml.

FIG. 5A-C: Chimeric human IFNγR-human IL12R (CCR) enhance human primary peripheral blood NK cell proliferation. CC12R transduced purified human primary NK cells from four donors (571-74) were cultured for 6 days with the indicated stimulation. CC12R expression was evaluated by flow cytometry using anti FLAG antibody (2^{nd} CC12R chain). Cell proliferation of live NK cells was assessed by labeling the cells with cell tracer violet (CTV, dye is diluted during cell division). Dead cells were excluded by using a near-infrared live/dead marker. Proliferation was evaluated relative to a matched control sample (CC12R−). FIG. 5A: CTV geometric mean fluorescence intensity (gMFI) levels in CC12R+ relative to CC12R− NK cells. IL-2^{low}=3 U/ml, IL-12^{low}=2.5 ng/ml. FIG. 5B: Percentage of CTV^{low} cells in CC12R+ relative to CC12R− NK cells. IL-2^{low}=3 U/ml, IL-12^{low}=2.5 ng/ml. FIG. 5C: Histograms showing CTV levels in CC12R+ relative to CC12R− NK cells. Paired t-test, parametric, *p<0.05, **p<0.01, ***p<0.001. Mean+/−S.D. A and B, human IL-12^{low}=2.5 ng/ml, human IL-2^{low}=3 U/ml; C, human IL-12=2.5 ng/ml.

FIG. 6A-B: CC12R enhances human primary NK cell function and proliferation. CC12R-transduced purified human primary peripheral blood NK cells from three donors were assessed for: (A) IFNγ secretion after 6 days of culture with human IL-2^{high} (300 U/ml), human IL-2^{low} (15 U/ml), human IL-18h^{igh} (25 ng/ml), human IL-18low (2.5 ng/ml), human IL-12 (2.5 ng/ml), mouse anti-NKp30 antibody (IgG1)-coated beads, or isotype-matched control mouse IgG1-coated beads in 96 U-shaped well plates. Purified antibody-conjugated beads were prepared according to the company's protocol (Invitrogen™ Dynabeads™ Antibody Coupling Kit) at 10 μg antibody per 1 mg beads. Anti-NKp30 (BioLegend cat. 325204, mouse IgG1k), mouse IgG1 isotype-matched control (clone; MOPC-21). Following conjugation, beads were resuspended in sterile phosphate-buffered saline at an antibody concentration of 0.1 μg/μl. Antibody conjugation was evaluated by flow cytometry with APC-conjugated anti-mouse or rat IgG. Antibody-conjugated beads were kept at 4° C. One μl antibody-coated beads were added to 500 μl media containing NK cells. Culture media was collected after 6 days, and IFNγ concentrations (pg/ml) was evaluated by ELISA. (B) NK cells absolute numbers following co-culture with irradiated 721.221-membrane-bound human IL-21 cells in 24-well G-Rex plate, 8 ml/well, effector to target ratio=1:10). NK cells were gated as live CD3− CD56+ NK cells. Dead cells were excluded by using a near-infrared live/dead dye. FIG. 6A: Human IFNγ concentrations. FIG. 6B: Absolute NK cell numbers. Paired t-test, one tail, *p<0.05, **p<0.01, ***p<0.001. Mean+/−S.D. CCR12+ relative to CC12R− donor-matched NK cells.

### DEFINITIONS

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The phrase “specifically (or selectively) binds” to a ligand or target, when referring to a protein or protein domain (e.g., portion), refers to a binding reaction whereby the protein binds to the ligand or target of interest. In the context of this disclosure, an ectodomain specifically binds a ligand with a K_{D} that is at least 100-fold stronger (lower value) than its affinity for other ligands (e.g., different unrelated ligands).

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region. Alignment for purposes of determining percentage amino acid sequence identity can be performed using publicly available computer software such as BLAST-2.0. The BLAST and BLAST 2.0 algorithm, are described in Altschul et al., Nuc. Acids Res. 25:3389-3402 (1977) and Altschul et al., J. Mol. Biol. 215:403-410 (1990). Thus, BLAST 2.0 can be used with the default parameters to determine percent sequence identity.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, or combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendant moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpin, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The terms “subject”, “patient” or “individual” are used herein interchangeably to refer to any mammal, including, but not limited to, a human. For example, the animal subject may be, a primate (e.g., a monkey, chimpanzee), a livestock animal (e.g., a horse, a cow, a sheep, a pig, or a goat), a companion animal (e.g., a dog, a cat), a laboratory test animal (e.g., a mouse, a rat, a guinea pig), or any other mammal. In some embodiments, the subject”, “patient” or “individual” is a human.

“Chimeric” as used herein refers to the fusion of two polypeptide sequences that do not occur in nature. For example a fusion of an ectodomain of a first receptor protein with the intracellular domain of a second receptor protein.

Natural killer cells, also known as NK cells, are a type of cytotoxic lymphocyte involved in the innate immune system. NK cells can be identified by the presence of CD56 and the absence of CD3 (CD56+,CD3−). See, e.g., Pfefferle A, et al., (2020). “Deciphering Natural Killer Cell Homeostasis”. Frontiers in Immunology. 11: 812; Schmidt S, et al., (2018). “Natural killer cells as a therapeutic tool for infectious diseases—current status and future perspectives”. Oncotarget. 9 (29): 20891-20907. CD94 is expressed primarily on NK cells (see, e.g., Guntauri et al., Immunol Res 30(1):29-34 (2004)).

The terms “therapeutically effective dose,” “effective dose,” or “therapeutically effective amount” herein is meant a dose that produces effects for which it is administered. The exact dose and formulation will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

The term “treat” and “treatment” refer to both therapeutic treatment and prophylactic or preventive measures, wherein the object is to prevent or slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

### DETAILED DESCRIPTION OF THE INVENTION

Natural Killer (NK) cells are innate lymphocytes with the ability to lyse tumor cells. One limitation of NK cells when encountering tumor cells is that they cannot control their own proliferation and expansion to increase the number of effector cells. During anti-viral immune responses, NK cells activate myeloid cells through the secretion of IFNγ and GM-CSF. Following stimulation by NK cells, myeloid cells can present IL-15 and/or secrete IL-12, which in turn can support proliferation and activation of NK cells. While this mechanism is robust in anti-viral immune responses, it is often feeble or non-existent at tumor sites. Moreover, administration of recombinant IL-15, IL-2, or IL-12 to patients has shown to have toxic effects, limiting their use to stimulate NK cell-mediated immunity against cancer cells in vivo. To promote autocrine growth signals for NK cells, the inventors have developed a chimeric cytokine receptor (CCR; which is a receptor formed from two different polypeptide strands, each strand being a chimeric transmembrane polypeptide) by fusion of the human IFNγ receptor ectodomains with the intracellular signaling domains of human IL-12 or human IL-15 (IL-2) receptors. These CCRs are able to support human NK cell line (NK92) growth in vitro directly by provision of IFNγ, not requiring the presence of IL-2, IL-15, or, IL-12. Additionally, a CCR, having IFNγ receptor-binding ectodomains, with IL-12 receptor intracellular domains, was expressed in human primary NK cells and showed increased NK cell sensitivity to exogenous IL-2, allowing for better NK cell proliferation at high or low IL-2 concentrations. In view of these discoveries, additional ectodomains and/or signaling domains can be used as described in more detail below to improve NK cell or T-cell activation and proliferation, including in their use against tumor cells.

Some advantages of the discoveries provided herein include, e.g., (i) reduced sensitivity of NK cells expressing a CCR to exogenous IL-2 stimulation; (ii) reduction or elimination of the need to expand primary human NK cells in vitro prior to infusion into a human; (iii) reduction or elimination of the need to systemically administer IL-12 to stimulate NK cells in a human, thereby reducing or eliminating toxic effects of systemic IL-12 administration; (iv) reduced toxic effects of systemic IFNγ secretion by NK cell activation that can be caused by systemic IL-12 or IL-2 administration; (v) increased in vivo expansion of primary human NK cells; (vi) increased primary human NK cell effector functions; and (vii) increased localization of IL-12-mediated anti-tumor effects through NK cells.

A variety of CCR configurations are provided herein that can be expressed in NK cells or T-cells (e.g., CD4+ or CD8+ T-cells). The CCRs involve a first and a second chimeric transmembrane polypeptide, each including one ectodomain and one signaling domain. The use of the words “first” and “second” are simply to distinguish the two polypeptides from each other. When the ectodomains from the first and second chimeric transmembrane polypeptides bind to the same ligand, the respective linked signaling domains of the chimeric transmembrane polypeptides are brought into proximity to generate a signal. For example, a variety of ectodomains (extracellular domains that bind to a ligand and which can bind the ligand in pairs or greater aggregates thereby bringing into proximity and activating linked intracellular signaling domains) can be used depending on which ligand is desired to activate the NK cells. A variety of signaling domains can be paired with the ectodomains via a linking transmembrane domain. Interleukin signaling domains in various pairings generate intracellular signaling. The CCRs described herein can be introduced into NK cells (e.g., via expression of introduced nucleic acids encoding the CCR) and the NK cells can be introduced into a human (e.g., having a tumor), whereby the NK cells ameliorate or treat the cancer.

As noted above, two or more different chimeric transmembrane polypeptides can be expressed in an NK cell, wherein each of the two chimeric transmembrane polypeptides have different ectodomains that bind to the same ligand. The identity of the ectodomains will depend on the ligand to act as the stimulus for bringing the two different chimeric transmembrane polypeptides in proximity. Extracellular portions of receptors having ligand-binding domains are generally known and can be readily assayed. In some embodiments, one can assay various fragments of an extracellular portion of a receptor to identify a minimal fragment having ligand binding activity.

In some embodiments, the ligand is a molecule produced (e.g., secreted) by the NK cells themselves, thereby resulting in a cycle of expression of the ligand and proliferation of the NK cells triggered by the expressed ligand. Non-limiting examples of such ligands can include, for example Interferon-gamma (IFNg or IFNγ) or GM-CSF. Alternatively, a ligand and respective ectodomains can be selected wherein the ligand is produced by other cells or that is provided exogenously. In one example, the ectodomains bind to TGF-β, which inhibits NK cells but in the context of binding a CCR as described herein, would stimulate activation or proliferation of the NK cells expressing the CCR.

In some embodiments, the first chimeric transmembrane polypeptide includes an IFNγ-binding ectodomain from human interferon-gamma receptor 1 (IFNGR1) and the second chimeric transmembrane polypeptide includes an IFNγ-binding ectodomain from human interferon-gamma receptor 2 (IFNGR2).

In some embodiments, the IFNγ-binding ectodomain from IFNGR1 comprises an amino acid sequence substantially (e.g., at least 90, 91, 92, 93, 94, 95, 96, 97, 98, 99 or 100%) identical to

(SEQ ID NO: 1)
EMGTADLGPSSVPTPTNVTIESYNMNPIVYWEYQIMPQVPVFTVEVKNY
GVKNSEWIDACINISHHYCNISDHVGDPSNSLWVRVKARVGQKESAYAK
SEEFAVCRDGKIGPPKLDIRKEEKQIMIDIFHPSVFVNGDEQEVDYDPE
TTCYIRVYNVYVRMNGSEIQYKILTQKEDDCDEIQCQLAIPVSSLNSQY
CVSAEGVLHVWGVTTEKSKEVCITIFNSSIKG.

In some embodiments, the IFNγ-binding ectodomain from IFNGR2 comprises an amino acid sequence substantially (e.g., at least 90, 91, 92, 93, 94, 95, 96, 97, 98, 99 or 100%) identical to

(SEQ ID NO: 2)
APPAQLTLETYQEWCNDSAATHDPLSQLPAPQHPKIRLYNAEQVLSWEP
VALSNSTRPVVYQVQFKYTDSKWFTADIMSIGVNCTQITATECDFTAAS
PSAGFPMDFNVTLRLRAELGALHSAWVTMPWFQHYRNVTVGPPENIEVT
PGEGSLIIRFSSPFDIADTSTAFFCYYVHYWEKGGIQQVKGPFRSNSIS
LDNLKPSRVYCLQVQAQLLWNKSNIFRVGHLSNISCYETMADASTELQ
Q.

In some embodiments, the first chimeric transmembrane polypeptide includes an GM-CSF-binding ectodomain from human Colony Stimulating Factor 2 Receptor Subunit Alpha (CSF2RA) and the second chimeric transmembrane polypeptide includes an GM-CSF-binding ectodomain from human Colony Stimulating Factor 2 Receptor Subunit Beta (CSF2RB).

In some embodiments, the GM-CSF-binding ectodomain from human CSF2RA comprises an amino acid sequence substantially (e.g., at least 90, 91, 92, 93, 94, 95, 96, 97, 98, 99 or 100%) identical to

(SEQ ID NO: 3)
MLLLVTSLLLCELPHPAFLHHHHHHLIPEKSDLRTVAPASSLNVRFDSR
TMNLSWDCQENTTFSKCFLTDKKNRVVEPRLSNNECSCTFREICLHEGV
TFEVHVNTSQRGFQQKLLYPNSGREGTAAQNFSCFIYNADLMNCTWARG
PTAPRDVQYFLYIRNSKRRREIRCPYYIQDSGTHVGCHLDNLSGLTSRN
YFLVNGTSREIGIQFFDSLLDTKKIERFNPPSNVTVRCNTTHCLVRWKQ
PRTYQKLSYLDFQYQLDVHRKNTQPGTENLLINVSGDLENRYNFPSSEP
RAKHSVKIRAADVRILNWSSWSEAIEFGSDDG.

In some embodiments, the GM-CSF-binding ectodomain from human CSF2RB comprises an amino acid sequence substantially (e.g., at least 90, 91, 92, 93, 94, 95, 96, 97, 98, 99 or 100%) identical to

(SEQ ID NO: 4)
WERSLAGAEETIPLQTLRCYNDYTSHITCRWADTQDAQRLVNVTLIRRV
NEDLLEPVSCDLSDDMPWSACPHPRCVPRRCVIPCQSFVVTDVDYFSFQ
PDRPLGTRLTVTLTQHVQPPEPRDLQISTDQDHFLLTWSVALGSPQSHW
LSPGDLEFEVVYKRLQDSWEDAAILLSNTSQATLGPEHLMPSSTYVARV
RTRLAPGSRLSGRPSKWSPEVCWDSQPGDEAQPQNLECFFDGAAVLSCS
WEVRKEVASSVSFGLFYKPSPDAGEEECSPVLREGLGSLHTRHHCQIPV
PDPATHGQYIVSVQPRRAEKHIKSSVNIQMAPPSLNVTKDGDSYSLRWE
TMKMRYEHIDHTFEIQYRKDTATWKDSKTETLQNAHSMALPALEPSTRY
WARVRVRTSRTGYNGIWSEWSEARSWDTESVLPMW.

In some embodiments, the first chimeric transmembrane polypeptide includes a TGFβ-binding ectodomain from human Transforming Growth Factor beta Receptor 1 (TGFBR1) and the second chimeric transmembrane polypeptide includes a TGFβ-binding ectodomain from human Transforming growth factor beta receptor 2 (TGFBR2).

In some embodiments, the human TGFBR1 comprises an amino acid sequence substantially (e.g., at least 90, 91, 92, 93, 94, 95, 96, 97, 98, 99 or 100%) identical to

(SEQ ID NO: 5)
AAAALLPGATALQCFCHLCTKDNFTCVTDGLCFVSVTETTDKVIHNSMC
IAEIDLIPRDRPFVCAPSSKTGSVTTTYCCNQDHCNKIELPTTVKSSPG
LGPVE.

In some embodiments, the TGFβ-binding ectodomain from human TGFBR2 comprises an amino acid sequence substantially (e.g., at least 90, 91, 92, 93, 94, 95, 96, 97, 98, 99 or 100%) identical to

(SEQ ID NO: 6)
TIPPHVQKSDVEMEAQKDEIICPSCNRTAHPLRHINNDMIVTDNNGAVK
FPQLCKFCDVRFSTCDNQKSCMSNCSITSICEKPQEVCVAVWRKNDENI
TLETVCHDPKLPYHDFILEDAASPKCIMKEKKKPGETFFMCSCSSDECN
DNIIFSEEYNTSNPDLLLVIFQ.

It will be appreciated that fragments of the above sequences can also be used provided they specifically bind to their respective ligand.

The ectodomains (e.g., as described above) will be linked, via at least a transmembrane domain to one or more intracellular domains. As the goal is to bring two different signaling domains in proximity as a function of ligand binding of corresponding ectodomains, in general either signaling domain can be linked to either ectodomain with the same effect. As a theoretically example, ectodomain 1 and ectodomain 2 can be linked to signaling domain 1 and signaling domain 2, respectively, or alternatively ectodomain 1 and ectodomain 2 can be linked to signaling domain 2 and signaling domain 1, respectively. Signaling domains 1 and 2 will be selected for their ability, when in proximity, to generate a desired signal. In some embodiments, the signaling domain from a receptor can include the whole intracellular portion of a receptor. Alternatively, a fragment of the intracellular portion of a receptor can be used. In the latter case, if signaling domains have not been previously identified, one can generate a series of fragments and test them for activity, e.g., in a cell-based assay.

In some embodiments, the first chimeric transmembrane polypeptide includes a signaling domain from human Interleukin 12 Receptor Subunit Beta 1 (IL12RB1) and the second chimeric transmembrane polypeptide signaling domain from human Interleukin 12 Receptor Subunit Beta 2 (IL12RB2). In some embodiments, the first chimeric transmembrane polypeptide includes a signaling domain from human IL12RB2 and the second chimeric transmembrane polypeptide signaling domain from human IL12RB1.

In some embodiments, the IL12RB1 signaling domain comprises an amino acid sequence substantially (e.g., at least 90, 91, 92, 93, 94, 95, 96, 97, 98, 99 or 100%) identical to

(SEQ ID NO: 7)
NRAARHLCPPLPTPCASSAIEFPGGKETWQWINPVDFQEEASLQEALVV
EMSWDKGERTEPLEKTELPEGAPELALDTELSLEDGDRCKAKM.

In some embodiments, the IL12RB2 signaling domain comprises an amino acid sequence substantially (e.g., at least 90, 91, 92, 93, 94, 95, 96, 97, 98, 99 or 100%) identical to

(SEQ ID NO: 8)
HYFQQKVFVLLAALRPQWCSREIPDPANSTCAKKYPIAEEKTQLPLDRL
LIDWPTPEDPEPLVISEVLHQVTPVFRHPPCSNWPQREKGIQGHQASEK
DMMHSASSPPPPRALQAESRQLVDLYKVLESRGSDPKPENPACPWTVLP
AGDLPTHDGYLPSNIDDLPSHEAPLADSLEELEPQHISLSVFPSSSLHP
LTFSCGDKLTLDQLKMRCDSLML*.

In some embodiments, the first chimeric transmembrane polypeptide includes a signaling domain from human Interleukin 2 Receptor Beta (IL2RB) and the second chimeric transmembrane polypeptide signaling domain from human Interleukin 2 Receptor Gamma (IL2RG). In some embodiments, the first chimeric transmembrane polypeptide includes a signaling domain from human IL2RG and the second chimeric transmembrane polypeptide signaling domain from human IL2RB.

In some embodiments, the IL2RB signaling domain comprises an amino acid sequence substantially (e.g., at least 90, 91, 92, 93, 94, 95, 96, 97, 98, 99 or 100%) identical to

(SEQ ID NO: 9)
NCRNTGPWLKKVLKCNTPDPSKFFSQLSSEHGGDVQKWLSSPFPSSSFS
PGGLAPEISPLEVLERDKVTQLLLQQDKVPEPASLSSNHSLTSCFTNQG
YFFFHLPDALEIEACQVYFTYDPYSEEDPDEGVAGAPTGSSPQPLQPLS
GEDDAYCTFPSRDDLLLFSPSLLGGPSPPSTAPGGSGAGEERMPPSLQE
RVPRDWDPQPLGPPTPGVPDLVDFQPPPELVLREAGEEVPDAGPREGVS
FPWSRPPGQGEFRALNARLPLNTDAYLSLQELQGQDPTHLV.

In some embodiments, the IL2RG signaling domain comprises an amino acid sequence substantially (e.g., at least 90, 91, 92, 93, 94, 95, 96, 97, 98, 99 or 100%) identical to

(SEQ ID NO: 10)
ERTMPRIPTLKNLEDLVTEYHGNFSAWSGVSKGLAESLQPDYSERLCLV
SEIPPKGGALGEGPGASPCNQHSPYWAPPCYTLKPET.

In some embodiments, the first chimeric transmembrane polypeptide includes a signaling domain from human IL12RB1 and the second chimeric transmembrane polypeptide signaling domain from human Interleukin-23 Receptor (IL23R). In some embodiments, the first chimeric transmembrane polypeptide includes a signaling domain from human IL23R and the second chimeric transmembrane polypeptide signaling domain from human IL12RB1.

In some embodiments, the IL23R signaling domain comprises an amino acid sequence substantially (e.g., at least 90, 91, 92, 93, 94, 95, 96, 97, 98, 99 or 100%) identical to

(SEQ ID NO: 11)
NRSFRTGIKRRILLLIPKWLYEDIPNMKNSNVVKMLQENSELMNNNSSE
QVLYVDPMITEIKEIFIPEHKPTDYKKENTGPLETRDYPQNSLFDNTTV
VYIPDLNTGYKPQISNFLPEGSHLSNNNEITSLTLKPPVDSLDSGNNPR
LQKHPNFAFSVSSVNSLSNTIFLGELSLILNQGECSSPDIQNSVEEETT
MLLENDSPSETIPEQTLLPDEFVSCLGIVNEELPSINTYFPQNILESHF
NRISLLEK.

In some embodiments, the first chimeric transmembrane polypeptide includes a signaling domain from human IL12RB2 and the second chimeric transmembrane polypeptide signaling domain from human Glycoprotein 130 (GP130). In some embodiments, the first chimeric transmembrane polypeptide includes a signaling domain from human GP130 and the second chimeric transmembrane polypeptide signaling domain from human IL12RB2.

In some embodiments, the GP130 signaling domain comprises an amino acid sequence substantially (e.g., at least 90, 91, 92, 93, 94, 95, 96, 97, 98, 99 or 100%) identical to

(SEQ ID NO: 12)
NKRDLIKKHIWPNVPDPSKSHIAQWSPHTPPRHNFNSKDQMYSDGNFTD
VSVVEIEANDKKPFPEDLKSLDLFKKEKINTEGHSSGIGGSSCMSSSRP
SISSSDENESSQNTSSTVQYSTVVHSGYRHQVPSVQVFSRSESTQPLLD
SEERPEDLQLVDHVDGGDGILPRQQYFKQNCSQHESSPDISHFERSKQV
SSVNEEDFVRLKQQISDHISQSCGSGQMKMFQEVSAADAFGPGTEGQVE
RFETVGMEAATDEGMPKSYLPQTVRQGGYMPQ.

In some embodiments, the first chimeric transmembrane polypeptide includes a signaling domain from human IL12RB2 and the second chimeric transmembrane polypeptide signaling domain from human Interleukin 27 Receptor Subunit Alpha (IL27RA). In some embodiments, the first chimeric transmembrane polypeptide includes a signaling domain from human IL27RA and the second chimeric transmembrane polypeptide signaling domain from human IL12RB2.

In some embodiments, the IL27RA signaling domain comprises an amino acid sequence substantially (e.g., at least 90, 91, 92, 93, 94, 95, 96, 97, 98, 99 or 100%) identical to

(SEQ ID NO: 13)
TSGRCYHLRHKVLPRWVWEKVPDPANSSSGQPHMEQVPEAQPLGDLPIL
EVEEMEPPPVMESSQPAQATAPLDSGYEKHFLPTPEELGLLGPPRPQVL
A.

In some embodiments, the first chimeric transmembrane polypeptide includes a signaling domain from human IL2RG and the second chimeric transmembrane polypeptide signaling domain from human Interleukin 21 Receptor (IL21R). In some embodiments, the first chimeric transmembrane polypeptide includes a signaling domain from human IL21R and the second chimeric transmembrane polypeptide signaling domain from human IL2RG.

In some embodiments, the IL21R signaling domain comprises an amino acid sequence substantially (e.g., at least 90, 91, 92, 93, 94, 95, 96, 97, 98, 99 or 100%) identical to

(SEQ ID NO: 14)
SLKTHPLWRLWKKIWAVPSPERFFMPLYKGCSGDFKKWVGAPFTGSSLE
LGPWSPEVPSTLEVYSCHPPRSPAKRLQLTELQEPAELVESDGVPKPSF
WPTAQNSGGSAYSEERDRPYGLVSIDTVTVLDAEGPCTWPCSCEDDGYP
ALDLDAGLEPSPGLEDPLLDAGTTVLSCGCVSAGSPGLGGPLGSLLDRL
KPPLADGEDWAGGLPWGGRSPGGVSESEAGSPLAGLDMDTFDSGFVGSD
CSSPVECDFTSPGDEGPPRSYLRQWVVIPPPLSSPGPQAS.

In some embodiments, the first chimeric transmembrane polypeptide includes a signaling domain from human IL2RG and the second chimeric transmembrane polypeptide signaling domain from human Interleukin 4 Receptor (IL4R). In some embodiments, the first chimeric transmembrane polypeptide includes a signaling domain from human IL4R and the second chimeric transmembrane polypeptide signaling domain from human IL2RG.

In some embodiments, the IL4R signaling domain comprises an amino acid sequence substantially (e.g., at least 90, 91, 92, 93, 94, 95, 96, 97, 98, 99 or 100%) identical to

(SEQ ID NO: 15)
KIKKEWWDQIPNPARSRLVAIIIQDAQGSQWEKRSRGQEPAKCPHWKNC
LTKLLPCFLEHNMKRDEDPHKAAKEMPFQGSGKSAWCPVEISKTVLWPE
SISVVRCVELFEAPVECEEEEEVEEEKGSFCASPESSRDDFQEGREGIV
ARLTESLFLDLLGEENGGFCQQDMGESCLLPPSGSTSAHMPWDEFPSAG
PKEAPPWGKEQPLHLEPSPPASPTQSPDNLTCTETPLVIAGNPAYRSFS
NSLSQSPCPRELGPDPLLARHLEEVEPEMPCVPQLSEPTTVPQPEPETW
EQILRRNVLQHGAAAAPVSAPTSGYQEFVHAVEQGGTQASAVVGLGPPG
EAGYKAFSSLLASSAVSPEKCGFGASSGEEGYKPFQDLIPGCPGDPAPV
PVPLFTFGLDREPPRSPQSSHLPSSSPEHLGLEPGEKVEDMPKPPLPQE
QATDPLVDSLGSGIVYSALTCHLCGHLKQCHGQEDGGQT.

In some embodiments, the first chimeric transmembrane polypeptide includes a signaling domain from human IL2RG and the second chimeric transmembrane polypeptide signaling domain from human Interleukin 7 Receptor (IL7R). In some embodiments, the first chimeric transmembrane polypeptide includes a signaling domain from human IL7R and the second chimeric transmembrane polypeptide signaling domain from human IL2RG.

In some embodiments, the IL7R signaling domain comprises an amino acid sequence substantially (e.g., at least 90, 91, 92, 93, 94, 95, 96, 97, 98, 99 or 100%) identical to

(SEQ ID NO: 16)
KKRIKPIVWPSLPDHKKTLEHLCKKPRKNLNVSFNPESFLDCQIHRVDD
IQARDEVEGFLQDTFPQQLEESEKQRLGGDVQSPNCPSEDVVITPESFG
RDSSLTCLAGNVSACDAPILSSSRSLDCRESGKNGPHVYQDLLLSLGTT
NSTLPPPFSLQSGILTLNPVAQGQPILTSLGSNQEEAYVTMSSFYQNQ.

In some embodiments, the first chimeric transmembrane polypeptide includes a signaling domain from human IL2RG and the second chimeric transmembrane polypeptide signaling domain from human Interleukin 9 Receptor (IL9R). In some embodiments, the first chimeric transmembrane polypeptide includes a signaling domain from human IL9R and the second chimeric transmembrane polypeptide signaling domain from human IL2RG.

In some embodiments, the IL9R signaling domain comprises an amino acid sequence substantially (e.g., at least 90, 91, 92, 93, 94, 95, 96, 97, 98, 99 or 100%) identical to

(SEQ ID NO: 17)
KLSPRVKRIFYQNVPSPAMFFQPLYSVHNGNFQTWMGAHGAGVLLSQDC
AGTPQGALEPCVQEATALLTCGPARPWKSVALEEEQEGPGTRLPGNLSS
EDVLPAGCTEWRVQTLAYLPQEDWAPTSLTRPAPPDSEGSRSSSSSSSS
NNNNYCALGCYGGWHLSALPGNTQSSGPIPALACGLSCDHQGLETQQGV
AWVLAGHCQRPGLHEDLQGMLLPSVLSKARSWTF.

In some embodiments, the first chimeric transmembrane polypeptide includes a signaling domain from human IL2RB and the second chimeric transmembrane polypeptide signaling domain from human IL12RB1. In some embodiments, the first chimeric transmembrane polypeptide includes a signaling domain from human IL12RB1 and the second chimeric transmembrane polypeptide signaling domain from human IL2RB.

In some embodiments, the first chimeric transmembrane polypeptide includes a signaling domain from human IL2RB and the second chimeric transmembrane polypeptide signaling domain from human IL12RB2. In some embodiments, the first chimeric transmembrane polypeptide includes a signaling domain from human IL12RB2 and the second chimeric transmembrane polypeptide signaling domain from human IL2RB.

In some embodiments, the first chimeric transmembrane polypeptide includes a signaling domain from human IL2RG and the second chimeric transmembrane polypeptide signaling domain from human IL12RB2. In some embodiments, the first chimeric transmembrane polypeptide includes a signaling domain from human IL12RB2 and the second chimeric transmembrane polypeptide signaling domain from human IL2RG.

In some embodiments, the first chimeric transmembrane polypeptide includes a signaling domain from human IL2RG and the second chimeric transmembrane polypeptide signaling domain from human IL12RB2. In some embodiments, the first chimeric transmembrane polypeptide includes a signaling domain from human IL12RB2 and the second chimeric transmembrane polypeptide signaling domain from human IL2RG.

As noted above, when the ectodomains of the first chimeric transmembrane polypeptide and the second chimeric transmembrane polypeptide bind the same ligand, their respective signaling domains are brought in proximity, thereby generating signaling activity. In some embodiments, the signaling activity is activation of the Janus kinase (JAK)-STAT (signal transducer and activator of transcription) pathway of signal transduction, whose activity can be measured in several ways as is known in the art. See, e.g., Murray, J Immunol Mar. 1, 2007, 178 (5) 2623-2629; Trinchieri, G. (2003). Nature Reviews Immunology, 3(2), 133-146; Liu, Z., et al. (2015). Frontiers in Cancer Immunology: Cancer Immunotherapy: Mechanisms of Cancer Immunity, Engineering Immune-Based Therapies and Developing Clinical Trials, 1, 91; Choi, J., et al. (2015). Clinical reviews in allergy & immunology, 49(3), 327-332; Sun, L., et al. (2015). Cytokine, 75(2), 249-255; Vignali, D. A., & Kuchroo, V. K. (2012). Nature immunology, 13(8), 722; Leonard et al., Nature Reviews Immunology volume 5, pages 688-698 (2005); Meazza, et al., BioMed Research International, vol. 2011, Article ID 861920, 16 pages, 2011. In some embodiments, the signaling is STAT3 signaling. In some embodiments, the signaling is STAT4 signaling. In some embodiments, the signaling is STAT5 signaling. In some embodiments, flow cytometry or western blotting is performed to measure the expression level of specific phosphorylated proteins associated with signal transduction and receptor function, such as pSTAT3, pSTAT4, or pSTAT5 signaling.

The ectodomain and the signaling domains in the chimeric transmembrane polypeptides are linked by at least a transmembrane domain, and optionally further linker sequences on either or both sides of the transmembrane domain. The transmembrane domain can be selected, for example, from any of a wide variety of transmembrane domains known in the art. In some embodiments, the transmembrane domain comprises any one of SEQ ID NOS: 18-32. In some embodiments, the transmembrane domain is selected to be from the same protein from which the ectodomain, or alternatively, the signaling domain, was obtained. As an example, in some embodiments, the IFNGR1-transmembrane domain can be in some embodiments used with the IFNGR1 ectodomain.

A non-limiting list of transmembrane sequences is listed below, which can be used with the ectodomain or signaling domain of the same origin or of different origin:

(SEQ ID NO: 18; IFNGR1-transmembrane domain)
SLWIPVVAALLLFLVLSLVFI
(SEQ ID NO: 19; IFNGR2-transmembrane domain)
VILISVGTFSLLSVLAGACFF
(SEQ ID NO: 20; IL 12R1-transmembrane domain)
WLIFFASLGSFLSILLVGVLGYLGL
(SEQ ID NO: 21; IL12R2-transmembrane domain)
WMAFVAPSICIAIIMVGIFST
(SEQ ID NO: 22; IL2RB-transmembrane domain)
IPWLGHLLVGLSGAFGFIILVYLLI
(SEQ ID NO: 23; IL2RG- transmembrane domain)
VVISVGSMGLIISLLCVYFWL
(SEQ ID NO: 24; CSF2RA transmembrane domain)
NLGSVYIYVLLIVGTLVCGIVLGFLF
(SEQ ID NO: 25; CSF2RB transmembrane domain)
VLALIVIFLTIAVLLAL
(SEQ ID NO: 26; IL23R transmembrane domain)
LLLGMIVFAVMLSILSLIGIF
(SEQ ID NO: 27; IL27R transmembrane domain)
VLPGILFLWGLFLLGCGLSLA
(SEQ ID NO: 28; GP130 transmembrane domain)
AIVVPVCLAFLLTTLLGVLFCF
(SEQ ID NO: 29; IL21R transmembrane domain)
GWNPHLLLLLLLVIVFIPAFW
(SEQ ID NO: 30; IL4R transmembrane domain)
LLLGVSVSCIVILAVCLLCYVSIT
(SEQ ID NO: 31; IL7R transmembrane domain)
PILLTISILSFFSVALLVILACVLW
(SEQ ID NO: 32; IL9R transmembrane domain)
GNTLVAVSIFLLLTGPTYLLF

Further, the chimeric transmembrane polypeptides described herein can include additional sequences at the amino or carboxyl terminus or within the sequence (e.g., linking two of the components of the polypeptide). In some embodiments, the polypeptides include a signal sequence, e.g., an amino terminal signal sequence, for example such that the protein is oriented properly in the cellular membrane. Exemplary signal sequences can include, for example, MALLFLLPLVMQGVSRA (IFNGR1 signal peptide; SEQ ID NO:33).

In some embodiments, the chimeric transmembrane polypeptides can include one or more epitope tag sequences that allow for convenient purification and tracking of the protein. Exemplary epitope tags for which specific monoclonal antibodies are readily available include, for example, FLAG (e.g., DYKDDDDK; SEQ ID NO: 34), influenza virus haemagglutinin (HA), and c-Myc tags (e.g., EQKLISEEDL; SEQ ID NO: 35).

Also provided are nucleic acids (e.g., DNA RNA) encoding the chimeric transmembrane polypeptides described herein, as well as host cells expressing the polypeptides. Because the chimeric transmembrane polypeptides are to be employed as pairs in a cell, in some embodiments the pair of chimeric transmembrane polypeptides are expressed as one larger fusion polypeptide linked via a cleavable peptide linker. Exemplary cleavable peptide sequences can include, but are not limited to a T2a peptide sequence (e.g., EGRGSLLTCGDVEENPGP; SEQ ID NO: 36) a P2A peptide (e.g., ATNFSLLKQAGDVEENPGP; SEQ ID NO: 37), an E2A peptide sequence (e.g., QCTNYALLKLAGDVESNPGP; SEQ ID NO: 38), or an F2A peptide sequence (e.g., VKQTLNFDLLKLAGDVESNPGP; SEQ ID NO: 39). The 2A peptide can undergo “self-cleavage” to generate mature proteins by a translational effect that is known as “stop-go” or “stop-carry” (Wang et al. (2015), Nature Scientific Reports 5:16237). Once expressed in the cells, the cleavable peptide sequence is cleaved, resulting in two separate proteins (i.e., a pair of the chimeric transmembrane polypeptides). In some embodiments, the cleavage sequence is targeted by a protease. For example, the protease furin targets specific sequences that can be used to separate two polypeptides. An exemplary furin cleavage sequence comprises (RKRR).

Exemplary protein sequences encoding a pair of chimeric transmembrane polypeptides separate by cleavable peptides sequences include but are not limited to the following (after the full sequence, each component sequence is listed in order):

IFNGR1-IL12RB1-IFNGR2-IL12RB2
MALLFLLPLVMQGVSRAEQKLISEEDLEMGTADLGPSSVPTPTNVTIESYNMNPIVYWE
YQIMPQVPVFTVEVKNYGVKNSEWIDACINISHHYCNISDHVGDPSNSLWVRVKARVG
QKESAYAKSEEFAVCRDGKIGPPKLDIRKEEKQIMIDIFHPSVFVNGDEQEVDYDPETTC
YIRVYNVYVRMNGSEIQYKILTQKEDDCDEIQCQLAIPVSSLNSQYCVSAEGVLHVWGV
TTEKSKEVCITIFNSSIKGSLWIPVVAALLLFLVLSLVFINRAARHLCPPLPTPCASSAIEFP
GGKETWQWINPVDFQEEASLQEALVVEMSWDKGERTEPLEKTELPEGAPELALDTELSL
EDGDRCKAKMRKRREGRGSLLTCGDVEENPGPMRPTLLWSLLLLLGVFAAAAADYKD
DDDKAPPAQLTLETYQEWCNDSAATHDPLSQLPAPQHPKIRLYNAEQVLSWEPVALSN
STRPVVYQVQFKYTDSKWFTADIMSIGVNCTQITATECDFTAASPSAGFPMDFNVTLRL
RAELGALHSAWVTMPWFQHYRNVTVGPPENIEVTPGEGSLIIRFSSPFDIADTSTAFFCY
YVHYWEKGGIQQVKGPFRSNSISLDNLKPSRVYCLQVQAQLLWNKSNIFRVGHLSNISC
YETMADASTELQQVILISVGTFSLLSVLAGACFFHYFQQKVFVLLAALRPQWCSREIPDP
ANSTCAKKYPIAEEKTQLPLDRLLIDWPTPEDPEPLVISEVLHQVTPVFRHPPCSNWPQRE
KGIQGHQASEKDMMHSASSPPPPRALQAESRQLVDLYKVLESRGSDPKPENPACPWTVL
PAGDLPTHDGYLPSNIDDLPSHEAPLADSLEELEPQHISLSVFPSSSLHPLTFSCGDKLTLD
QLKMRCDSLML (SEQ ID NO: 40)
1. MALLFLLPLVMQGVSRA (IFNGR1 signal peptide) (SEQ ID NO: 45)
2. EQKLISEEDL (Myc-tag) (SEQ ID NO: 46)
3. EMGTADLGPSSVPTPTNVTIESYNMNPIVYWEYQIMPQVPVFTVEVKNYGVKNS
EWIDACINISHHYCNISDHVGDPSNSLWVRVKARVGQKESAYAKSEEFAVCRDG
KIGPPKLDIRKEEKQIMIDIFHPSVFVNGDEQEVDYDPETTCYIRVYNVYVRMNG
SEIQYKILTQKEDDCDEIQCQLAIPVSSLNSQYCVSAEGVLHVWGVTTEKSKEVCI
TIFNSSIKG (IFNGR 1-ectodomain) (SEQ ID NO: 47)
4. SLWIPVVAALLLFLVLSLVFI (IFNGR1-transmembrane domain) (SEQ ID NO: 48)
5. NRAARHLCPPLPTPCASSAIEFPGGKETWQWINPVDFQEEASLQEALVVEMSWD
KGERTEPLEKTELPEGAPELALDTELSLEDGDRCKAKM (IL 12RB 1- intracellular
domain) (SEQ ID NO: 49)
6. RKRR (Furin) (SEQ ID NO: 50)
7. EGRGSLLTCGDVEENPGP (T2A) or E2A, F2A, P2A) (SEQ ID NO: 51)
8. MRPTLLWSLLLLLGVFAAAAA (IFNGR2 signal peptide) (SEQ ID NO: 52)
9. DYKDDDDK (FLAG-tag) (SEQ ID NO: 53)
10. APPAQLTLETYQEWCNDSAATHDPLSQLPAPQHPKIRLYNAEQVLSWEPVALSN
STRPVVYQVQFKYTDSKWFTADIMSIGVNCTQITATECDFTAASPSAGFPMDFNV
TLRLRAELGALHSAWVTMPWFQHYRNVTVGPPENIEVTPGEGSLIIRFSSPFDIAD
TSTAFFCYYVHYWEKGGIQQVKGPFRSNSISLDNLKPSRVYCLQVQAQLLWNKS
NIFRVGHLSNISCYETMADASTELQQ (IFNGR2-ectodomain) (SEQ ID NO: 54)
11. VILISVGTFSLLSVLAGACFF (IFNGR2-transmembrane domain) (SEQ ID NO: 55)
12. HYFQQKVFVLLAALRPQWCSREIPDPANSTCAKKYPIAEEKTQLPLDRLLIDWPT
PEDPEPLVISEVLHQVTPVFRHPPCSNWPQREKGIQGHQASEKDMMHSASSPPPP
RALQAESRQLVDLYKVLESRGSDPKPENPACPWTVLPAGDLPTHDGYLPSNIDD
LPSHEAPLADSLEELEPQHISLSVFPSSSLHPLTFSCGDKLTLDQLKMRCDSLML*
(IL 12RB2- intracellular domain) (SEQ ID NO: 56)
IFNGR1-IL12RB2-IFNGR2-IL12RB1
MALLFLLPLVMQGVSRAEQKLISEEDLEMGTADLGPSSVPTPTNVTIESYNMNPIVYWE
YQIMPQVPVFTVEVKNYGVKNSEWIDACINISHHYCNISDHVGDPSNSLWVRVKARVG
QKESAYAKSEEFAVCRDGKIGPPKLDIRKEEKQIMIDIFHPSVFVNGDEQEVDYDPETTC
YIRVYNVYVRMNGSEIQYKILTQKEDDCDEIQCQLAIPVSSLNSQYCVSAEGVLHVWGV
TTEKSKEVCITIFNSSIKGSLWIPVVAALLLFLVLSLVFIHYFQQKVFVLLAALRPQWCSR
EIPDPANSTCAKKYPIAEEKTQLPLDRLLIDWPTPEDPEPLVISEVLHQVTPVFRHPPCSN
WPQREKGIQGHQASEKDMMHSASSPPPPRALQAESRQLVDLYKVLESRGSDPKPENPA
CPWTVLPAGDLPTHDGYLPSNIDDLPSHEAPLADSLEELEPQHISLSVFPSSSLHPLTFSCG
DKLTLDQLKMRCDSLMLRKRREGRGSLLTCGDVEENPGPMRPTLLWSLLLLLGVFAAA
AADYKDDDDKAPPAQLTLETYQEWCNDSAATHDPLSQLPAPQHPKIRLYNAEQVLSWE
PVALSNSTRPVVYQVQFKYTDSKWFTADIMSIGVNCTQITATECDFTAASPSAGFPMDF
NVTLRLRAELGALHSAWVTMPWFQHYRNVTVGPPENIEVTPGEGSLIIRFSSPFDIADTS
TAFFCYYVHYWEKGGIQQVKGPFRSNSISLDNLKPSRVYCLQVQAQLLWNKSNIFRVG
HLSNISCYETMADASTELQQVILISVGTFSLLSVLAGACFFNRAARHLCPPLPTPCASSAIE
FPGGKETWQWINPVDFQEEASLQEALVVEMSWDKGERTEPLEKTELPEGAPELALDTEL
SLEDGDRCKAKM (SEQ ID NO: 41)
1. MALLFLLPLVMQGVSRA (IFNGR1 signal peptide) (SEQ ID NO: 57)
2. EQKLISEEDL (Myc-tag) (SEQ ID NO: 58)
3. EMGTADLGPSSVPTPTNVTIESYNMNPIVYWEYQIMPQVPVFTVEVKNYGVKNS
EWIDACINISHHYCNISDHVGDPSNSLWVRVKARVGQKESAYAKSEEFAVCRDG
KIGPPKLDIRKEEKQIMIDIFHPSVFVNGDEQEVDYDPETTCYIRVYNVYVRMNG
SEIQYKILTQKEDDCDEIQCQLAIPVSSLNSQYCVSAEGVLHVWGVTTEKSKEVCI
TIFNSSIKG (IFNGR1-ectodomain) (SEQ ID NO: 59)
4. SLWIPVVAALLLFLVLSLVFI (IFNGR1-transmembrane domain) (SEQ ID NO: 60)
5. RKRR (Furin) (SEQ ID NO: 61)
6. HYFQQKVFVLLAALRPQWCSREIPDPANSTCAKKYPIAEEKTQLPLDRLLIDWPT
PEDPEPLVISEVLHQVTPVFRHPPCSNWPQREKGIQGHQASEKDMMHSASSPPPP
RALQAESRQLVDLYKVLESRGSDPKPENPACPWTVLPAGDLPTHDGYLPSNIDD
LPSHEAPLADSLEELEPQHISLSVFPSSSLHPLTFSCGDKLTLDQLKMRCDSLML
(IL12RB2-intracellular domain) (SEQ ID NO: 62)
7. EGRGSLLTCGDVEENPGP (T2A) or E2A, F2A, P2A) (SEQ ID NO: 63)
8. MRPTLLWSLLLLLGVFAAAAA (IFNGR2 signal peptide) (SEQ ID NO: 64)
9. DYKDDDDK (FLAG-tag) (SEQ ID NO: 65)
10. APPAQLTLETYQEWCNDSAATHDPLSQLPAPQHPKIRLYNAEQVLSWEPVALSN
STRPVVYQVQFKYTDSKWFTADIMSIGVNCTQITATECDFTAASPSAGFPMDFNV
TLRLRAELGALHSAWVTMPWFQHYRNVTVGPPENIEVTPGEGSLIIRFSSPFDIAD
TSTAFFCYYVHYWEKGGIQQVKGPFRSNSISLDNLKPSRVYCLQVQAQLLWNKS
NIFRVGHLSNISCYETMADASTELQQ (IFNGR2-ectodomain) (SEQ ID NO: 66)
11. VILISVGTFSLLSVLAGACFF (IFNGR2-transmembrane domain) (SEQ ID NO: 67)
12. NRAARHLCPPLPTPCASSAIEFPGGKETWQWINPVDFQEEASLQEALVVEMSWD
KGERTEPLEKTELPEGAPELALDTELSLEDGDRCKAKM* (IL12RB1-intracellular
domain) (SEQ ID NO: 68)
IFNGR1-IL12RB1-IFNGR2-IL12RB2
MALLFLLPLVMQGVSRAEQKLISEEDLEMGTADLGPSSVPTPTNVTIESYNMNPIVYWE
YQIMPQVPVFTVEVKNYGVKNSEWIDACINISHHYCNISDHVGDPSNSLWVRVKARVG
QKESAYAKSEEFAVCRDGKIGPPKLDIRKEEKQIMIDIFHPSVFVNGDEQEVDYDPETTC
YIRVYNVYVRMNGSEIQYKILTQKEDDCDEIQCQLAIPVSSLNSQYCVSAEGVLHVWGV
TTEKSKEVCITIFNSSIKGWLIFFASLGSFLSILLVGVLGYLGLNRAARHLCPPLPTPCASS
AIEFPGGKETWQWINPVDFQEEASLQEALVVEMSWDKGERTEPLEKTELPEGAPELALD
TELSLEDGDRCKAKMRKRREGRGSLLTCGDVEENPGPMRPTLLWSLLLLLGVFAAAAA
DYKDDDDKAPPAQLTLETYQEWCNDSAATHDPLSQLPAPQHPKIRLYNAEQVLSWEPV
ALSNSTRPVVYQVQFKYTDSKWFTADIMSIGVNCTQITATECDFTAASPSAGFPMDFNV
TLRLRAELGALHSAWVTMPWFQHYRNVTVGPPENIEVTPGEGSLIIRFSSPFDIADTSTAF
FCYYVHYWEKGGIQQVKGPFRSNSISLDNLKPSRVYCLQVQAQLLWNKSNIFRVGHLS
NISCYETMADASTELQQWMAFVAPSICIAIIMVGIFSTHYFQQKVFVLLAALRPQWCSRE
IPDPANSTCAKKYPIAEEKTQLPLDRLLIDWPTPEDPEPLVISEVLHQVTPVFRHPPCSNW
PQREKGIQGHQASEKDMMHSASSPPPPRALQAESRQLVDLYKVLESRGSDPKPENPACP
WTVLPAGDLPTHDGYLPSNIDDLPSHEAPLADSLEELEPQHISLSVFPSSSLHPLTFSCGD
KLTLDQLKMRCDSLML (SEQ ID NO: 42)
1. MALLFLLPLVMQGVSRA (IFNGR1 signal peptide) (SEQ ID NO: 69)
2. EQKLISEEDL (Myc-tag) (SEQ ID NO: 70)
3. EMGTADLGPSSVPTPTNVTIESYNMNPIVYWEYQIMPQVPVFTVEVKNYGVKNS
EWIDACINISHHYCNISDHVGDPSNSLWVRVKARVGQKESAYAKSEEFAVCRDG
KIGPPKLDIRKEEKQIMIDIFHPSVFVNGDEQEVDYDPETTCYIRVYNVYVRMNG
SEIQYKILTQKEDDCDEIQCQLAIPVSSLNSQYCVSAEGVLHVWGVTTEKSKEVCI
TIFNSSIKG (IFNGR1-ectodomain) (SEQ ID NO: 71)
4. WLIFFASLGSFLSILLVGVLGYLGL (IL12R1-transmembrane domain) (SEQ ID
NO: 72)
5. NRAARHLCPPLPTPCASSAIEFPGGKETWQWINPVDFQEEASLQEALVVEMSWD
KGERTEPLEKTELPEGAPELALDTELSLEDGDRCKAKM (IL12RB1-intracellular
domain) (SEQ ID NO: 73)
6. RKRR (Furin) (SEQ ID NO: 74)
7. EGRGSLLTCGDVEENPGP (T2A) or E2A, F2A, P2A) (SEQ ID NO: 75)
8. MRPTLLWSLLLLLGVFAAAAA (IFNGR2 signal peptide) (SEQ ID NO: 76)
9. DYKDDDDK (FLAG-tag) (SEQ ID NO: 77)
10. APPAQLTLETYQEWCNDSAATHDPLSQLPAPQHPKIRLYNAEQVLSWEPVALSN
STRPVVYQVQFKYTDSKWFTADIMSIGVNCTQITATECDFTAASPSAGFPMDFNV
TLRLRAELGALHSAWVTMPWFQHYRNVTVGPPENIEVTPGEGSLIIRFSSPFDIAD
TSTAFFCYYVHYWEKGGIQQVKGPFRSNSISLDNLKPSRVYCLQVQAQLLWNKS
NIFRVGHLSNISCYETMADASTELQQ (IFNGR2-ectodomain) (SEQ ID NO: 78)
11. WMAFVAPSICIAIIMVGIFST (IL12R2-transmembrane domain) (SEQ ID NO: 79)
12. HYFQQKVFVLLAALRPQWCSREIPDPANSTCAKKYPIAEEKTQLPLDRLLIDWPT
PEDPEPLVISEVLHQVTPVFRHPPCSNWPQREKGIQGHQASEKDMMHSASSPPPP
RALQAESRQLVDLYKVLESRGSDPKPENPACPWTVLPAGDLPTHDGYLPSNIDD
LPSHEAPLADSLEELEPQHISLSVFPSSSLHPLTFSCGDKLTLDQLKMRCDSLML*
(IL 12RB2-intracellular domain) (SEQ ID NO: 80)
IFNGRI-IL12RB2-IFNGR2-IL12RB1
MALLFLLPLVMQGVSRAEQKLISEEDLEMGTADLGPSSVPTPTNVTIESYNMNPIVYWE
YQIMPQVPVFTVEVKNYGVKNSEWIDACINISHHYCNISDHVGDPSNSLWVRVKARVG
QKESAYAKSEEFAVCRDGKIGPPKLDIRKEEKQIMIDIFHPSVFVNGDEQEVDYDPETTC
YIRVYNVYVRMNGSEIQYKILTQKEDDCDEIQCQLAIPVSSLNSQYCVSAEGVLHVWGV
TTEKSKEVCITIFNSSIKGWMAFVAPSICIAIIMVGIFSTHYFQQKVFVLLAALRPQWCSRE
IPDPANSTCAKKYPIAEEKTQLPLDRLLIDWPTPEDPEPLVISEVLHQVTPVFRHPPCSNW
PQREKGIQGHQASEKDMMHSASSPPPPRALQAESRQLVDLYKVLESRGSDPKPENPACP
WTVLPAGDLPTHDGYLPSNIDDLPSHEAPLADSLEELEPQHISLSVFPSSSLHPLTFSCGD
KLTLDQLKMRCDSLMLRKRREGRGSLLTCGDVEENPGPMRPTLLWSLLLLLGVFAAAA
ADYKDDDDKAPPAQLTLETYQEWCNDSAATHDPLSQLPAPQHPKIRLYNAEQVLSWEP
VALSNSTRPVVYQVQFKYTDSKWFTADIMSIGVNCTQITATECDFTAASPSAGFPMDFN
VTLRLRAELGALHSAWVTMPWFQHYRNVTVGPPENIEVTPGEGSLIIRFSSPFDIADTST
AFFCYYVHYWEKGGIQQVKGPFRSNSISLDNLKPSRVYCLQVQAQLLWNKSNIFRVGH
LSNISCYETMADASTELQQWLIFFASLGSFLSILLVGVLGYLGLNRAARHLCPPLPTPCAS
SAIEFPGGKETWQWINPVDFQEEASLQEALVVEMSWDKGERTEPLEKTELPEGAPELAL
DTELSLEDGDRCKAKM (SEQ ID NO: 43)
1. MALLFLLPLVMQGVSRA (IFNGR1 signal peptide) (SEQ ID NO: 81)
2. EQKLISEEDL (Myc-tag) (SEQ ID NO: 82)
3. EMGTADLGPSSVPTPTNVTIESYNMNPIVYWEYQIMPQVPVFTVEVKNYGVKNSEWIDA
CINISHHYCNISDHVGDPSNSLWVRVKARVGQKESAYAKSEEFAVCRDGKIGPPKLDIRK
EEKQIMIDIFHPSVFVNGDEQEVDYDPETTCYIRVYNVYVRMNGSEIQYKILTQKEDDCD
EIQCQLAIPVSSLNSQYCVSAEGVLHVWGVTTEKSKEVCITIFNSSIKG (IFNGR1-
ectodomain) (SEQ ID NO: 83)
4. WMAFVAPSICIAIIMVGIFST (IL12RB2-transmembrane domain) (SEQ ID NO: 84)
5. HYFQQKVFVLLAALRPQWCSREIPDPANSTCAKKYPIAEEKTQLPLDRLLIDWPTPEDPEP
LVISEVLHQVTPVFRHPPCSNWPQREKGIQGHQASEKDMMHSASSPPPPRALQAESRQLV
DLYKVLESRGSDPKPENPACPWTVLPAGDLPTHDGYLPSNIDDLPSHEAPLADSLEELEP
QHISLSVFPSSSLHPLTFSCGDKLTLDQLKMRCDSLML (IL12RB2-intracellular domain)
(SEQ ID NO: 85)
6. RKRR (Furin) (SEQ ID NO: 86)
7. EGRGSLLTCGDVEENPGP (T2A) or E2A, F2A, P2A) (SEQ ID NO: 87)
8. MRPTLLWSLLLLLGVFAAAAA (IFNGR2 signal peptide) (SEQ ID NO: 88)
9. DYKDDDDK (FLAG-tag) (SEQ ID NO: 89)
10. APPAQLTLETYQEWCNDSAATHDPLSQLPAPQHPKIRLYNAEQVLSWEPVALSNSTRPV
VYQVQFKYTDSKWFTADIMSIGVNCTQITATECDFTAASPSAGFPMDFNVTLRLRAELG
ALHSAWVTMPWFQHYRNVTVGPPENIEVTPGEGSLIIRFSSPFDIADTSTAFFCYYVHYW
EKGGIQQVKGPFRSNSISLDNLKPSRVYCLQVQAQLLWNKSNIFRVGHLSNISCYETMAD
ASTELQQ (IFNGR2-ectodomain) (SEQ ID NO: 90)
11. WLIFFASLGSFLSILLVGVLGYLGL (IL12RB1-transmembrane domain) (SEQ ID NO: 91)
12. NRAARHLCPPLPTPCASSAIEFPGGKETWQWINPVDFQEEASLQEALVVEMSWDKGERT
EPLEKTELPEGAPELALDTELSLEDGDRCKAKM* (IL12RB1-intracellular domain) (SEQ
ID NO: 92)
IFNGR1-IL2RB-IFNGR2-IL2RG
MALLFLLPLVMQGVSRAEQKLISEEDLEMGTADLGPSSVPTPTNVTIESYNMNPIVYWE
YQIMPQVPVFTVEVKNYGVKNSEWIDACINISHHYCNISDHVGDPSNSLWVRVKARVG
QKESAYAKSEEFAVCRDGKIGPPKLDIRKEEKQIMIDIFHPSVFVNGDEQEVDYDPETTC
YIRVYNVYVRMNGSEIQYKILTQKEDDCDEIQCQLAIPVSSLNSQYCVSAEGVLHVWGV
TTEKSKEVCITIFNSSIKGIPWLGHLLVGLSGAFGFIILVYLLINCRNTGPWLKKVLKCNTP
DPSKFFSQLSSEHGGDVQKWLSSPFPSSSFSPGGLAPEISPLEVLERDKVTQLLLQQDKVP
EPASLSSNHSLTSCFTNQGYFFFHLPDALEIEACQVYFTYDPYSEEDPDEGVAGAPTGSSP
QPLQPLSGEDDAYCTFPSRDDLLLFSPSLLGGPSPPSTAPGGSGAGEERMPPSLQERVPRD
WDPQPLGPPTPGVPDLVDFQPPPELVLREAGEEVPDAGPREGVSFPWSRPPGQGEFRAL
NARLPLNTDAYLSLQELQGQDPTHLVRKRREGRGSLLTCGDVEENPGPMRPTLLWSLLL
LLGVFAAAAADYKDDDDKAPPAQLTLETYQEWCNDSAATHDPLSQLPAPQHPKIRLYN
AEQVLSWEPVALSNSTRPVVYQVQFKYTDSKWFTADIMSIGVNCTQITATECDFTAASP
SAGFPMDFNVTLRLRAELGALHSAWVTMPWFQHYRNVTVGPPENIEVTPGEGSLIIRFSS
PFDIADTSTAFFCYYVHYWEKGGIQQVKGPFRSNSISLDNLKPSRVYCLQVQAQLLWNK
SNIFRVGHLSNISCYETMADASTELQQVVISVGSMGLIISLLCVYFWLERTMPRIPTLKNLE
DLVTEYHGNFSAWSGVSKGLAESLQPDYSERLCLVSEIPPKGGALGEGPGASPCNQHSPYWA
PPCYTLKPET (SEQ ID NO: 44)
1. MALLFLLPLVMQGVSRA (IFNGR1 signal peptide) (SEQ ID NO: 93)
2. EQKLISEEDL (Myc-tag) (SEQ ID NO: 94)
3. EMGTADLGPSSVPTPTNVTIESYNMNPIVYWEYQIMPQVPVFTVEVKNYGVKNS
EWIDACINISHHYCNISDHVGDPSNSLWVRVKARVGQKESAYAKSEEFAVCRDG
KIGPPKLDIRKEEKQIMIDIFHPSVFVNGDEQEVDYDPETTCYIRVYNVYVRMNG
SEIQYKILTQKEDDCDEIQCQLAIPVSSLNSQYCVSAEGVLHVWGVTTEKSKEVCI
TIFNSSIKG (IFNGR 1-ectodomain) (SEQ ID NO: 95)
4. IPWLGHLLVGLSGAFGFIILVYLLI (IL2RB-transmembrane domain) (SEQ ID
NO: 96)
5. NCRNTGPWLKKVLKCNTPDPSKFFSQLSSEHGGDVQKWLSSPFPSSSFSPGGLAP
EISPLEVLERDKVTQLLLQQDKVPEPASLSSNHSLTSCFTNQGYFFFHLPDALEIEA
CQVYFTYDPYSEEDPDEGVAGAPTGSSPQPLQPLSGEDDAYCTFPSRDDLLLFSPS
LLGGPSPPSTAPGGSGAGEERMPPSLQERVPRDWDPQPLGPPTPGVPDLVDFQPP
PELVLREAGEEVPDAGPREGVSFPWSRPPGQGEFRALNARLPLNTDAYLSLQELQ
GQDPTHLV (IL2RB-intracellular domain) (SEQ ID NO: 97)
6. RKRR (Furin) (SEQ ID NO: 98)
7. EGRGSLLTCGDVEENPGP (T2A) or E2A, F2A, P2A) (SEQ ID NO: 99)
8. MRPTLLWSLLLLLGVFAAAAA (IFNGR2 signal peptide) (SEQ ID NO: 100)
9. DYKDDDDK (FLAG-tag) (SEQ ID NO: 101)
10. APPAQLTLETYQEWCNDSAATHDPLSQLPAPQHPKIRLYNAEQVLSWEPVALSN
STRPVVYQVQFKYTDSKWFTADIMSIGVNCTQITATECDFTAASPSAGFPMDFNV
TLRLRAELGALHSAWVTMPWFQHYRNVTVGPPENIEVTPGEGSLIIRFSSPFDIAD
TSTAFFCYYVHYWEKGGIQQVKGPFRSNSISLDNLKPSRVYCLQVQAQLLWNKS
NIFRVGHLSNISCYETMADASTELQQ (IFNGR2-ectodomain) (SEQ ID NO: 102)
11. VVISVGSMGLIISLLCVYFWL (IL2RG-transmembrane domain) (SEQ ID NO: 103)
12. ERTMPRIPTLKNLEDLVTEYHGNFSAWSGVSKGLAESLQPDYSERLCLVSEIPPK
GGALGEGPGASPCNQHSPYWAPPCYTLKPET (IL2RG-intracellular domain) (SEQ
ID NO: 104)

Non-limiting examples of suitable methods for introducing a nucleic acid into a cell include electroporation (e.g., nucleofection), viral transduction, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. In some embodiments, a polynucleotide encoding a protein is delivered to the cell by a vector. For example, in some embodiments, the vector is a viral vector. Exemplary viral vectors can include, but are not limited to, adenoviral vectors, adeno-associated viral (AAV) vectors, and lentiviral vectors. Codon selection for the encoded proteins can be performed as known in the art. For example, the coding sequence can be codon-optimized for expression in human cells.

In some embodiments, genetic modification is performed using transposase-based systems for gene integration, CRISPR/Cas-mediated gene integration, TALENS or Zinc-finger nucleases integration techniques to introduce the nucleic acids into a cell. As an example, integration of the nucleic acids into a cell can be achieved using CRISPR-Cas9-mediated homologous recombination (see, e.g., Oed, et al., Cancers (Basel) 2020 June; 12(6): 1704).

Cells comprising the nucleic acids encoding the chimeric transmembrane polypeptide, or a pair thereof, can include, for example, mammalian cells. In some embodiments, the cells are human cells. In some embodiments, the cells are human natural killer cells. In some embodiments, the cells are human T cells.

### NK and T-Cells

Natural killer cells or T-cells expressing pairs of chimeric transmembrane polypeptides can be introduced into a human subject. Natural killer cells or T-cells can be obtained from the human subject (in which case the cells are autologous) or the natural killer cells or T-cells can be obtained from a separate human (in which case the cells are allogeneic). In some embodiments, the natural killer cells or T-cells can be generated from induced pluripotent stem cells. See, e.g., Cichocki, et al., Sci Transl Med. 2020 Nov. 4; 12(568); Minagawa, A., et al., (2018). Cell Stem Cell, 23(6), 850-858; Zeng, J., et al., Stem Cell Reports, 9(6), 1796-1812. In some allogeneic embodiments, the human from whom the NK cells or T-cells are obtained and the subject to receive the NK cells or T-cells have been selected based on HLA matching to reduce or avoid host rejection of the allogeneic NK cells or T-cells. In autologous or allogeneic embodiments, the natural killer cells or T-cells can be obtained and optionally enriched for from, for example a blood sample or cord blood from the human or generated from iPSC cells. Enrichment can comprise, for example, use of one or more specific antibody that binds to a target antigen on the surface of natural killer cells or T-cells, allowing for enrichment for natural killer cells or T-cells. In some embodiments, the target antigen is specifically expressed on natural killer cells or T-cells. In other embodiments, the target antigen can also be expressed on some other cells, and in some embodiments to a lesser extent than expressed on natural killer cells or T-cells. In some embodiments, cell sorting (e.g., flow cytometry or antibody-coated magnetic bead selection) can be used to enrich for the natural killer cells or T-cells.

The primary natural killer cells or T-cells obtained from the human can be modified to express the pair of chimeric transmembrane polypeptides (e.g., as described above or elsewhere herein) and then be administered to the human subject. Optionally, the natural killer cells or T-cells can be expanded to generate a larger number of natural killer cells or T-cells. Optionally the natural killer cells or T-cells can be enriched for those expressing at least one of or the pair of chimeric transmembrane polypeptides.

The natural killer cells or T-cells can be administered to the human subject via a suitable route, such as intravenous or intra-tumor administration (see, e.g., Liu et al., N Engl J Med. 2020 Feb. 6; 382(6):545-553). Human subjects can be treated by infusing therapeutically effective doses of the NK cells or T-cells is in the range of about 10⁵ to 10¹⁰ or more cells per kilogram of body weight (cells/kg). The infusion can be repeated as often and as many times as the subject can tolerate until the desired response is achieved. The appropriate infusion dose and schedule will vary from subject to subject, but can be determined by the treating physician for a particular patient.

Subjects receiving NK cells or T-cells expressing a CCR pair as described herein can include subjects who have been diagnosed with cancer. Depending on whether or not tumors can spread by invasion and metastasis, they are classified as being either benign or malignant: benign tumors are tumors that cannot spread by invasion or metastasis, i.e., they only grow locally; whereas malignant tumors are tumors that are capable of spreading by invasion and metastasis. The methods described herein are useful for the treatment of local and malignant tumors. Exemplary types of cancer include, but are not limited to: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyo sarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers are-known to one of ordinary skill in the art.

### EXAMPLES

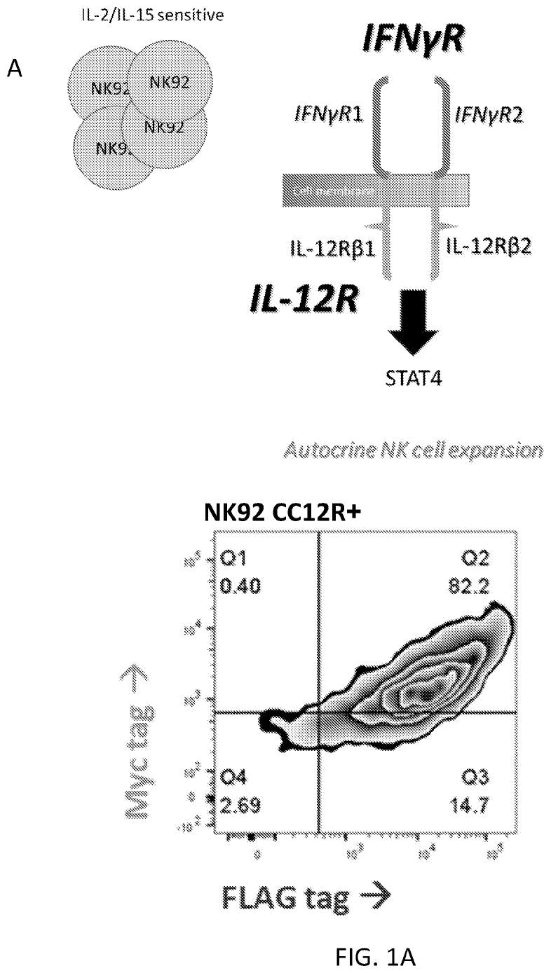

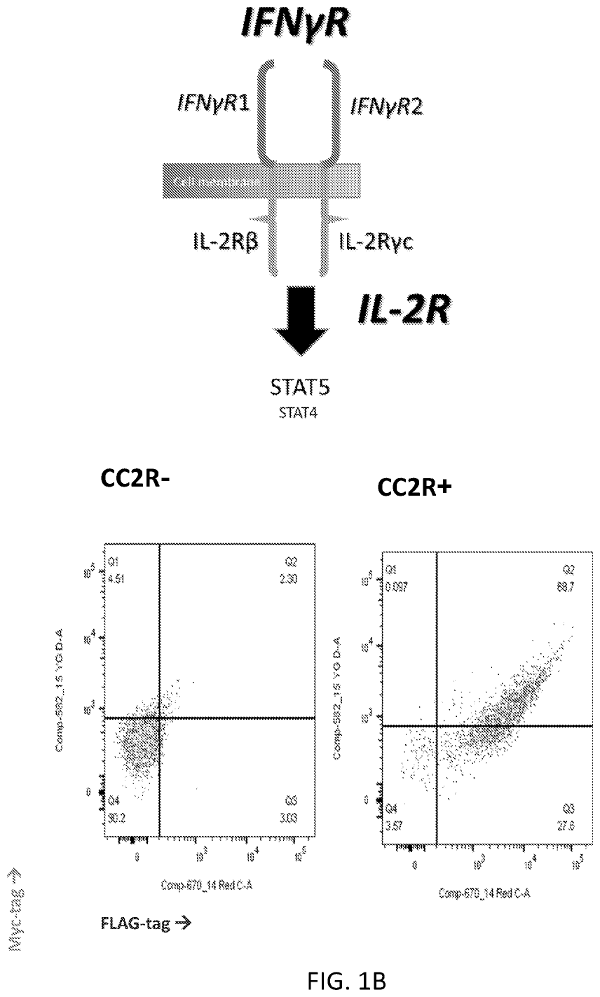

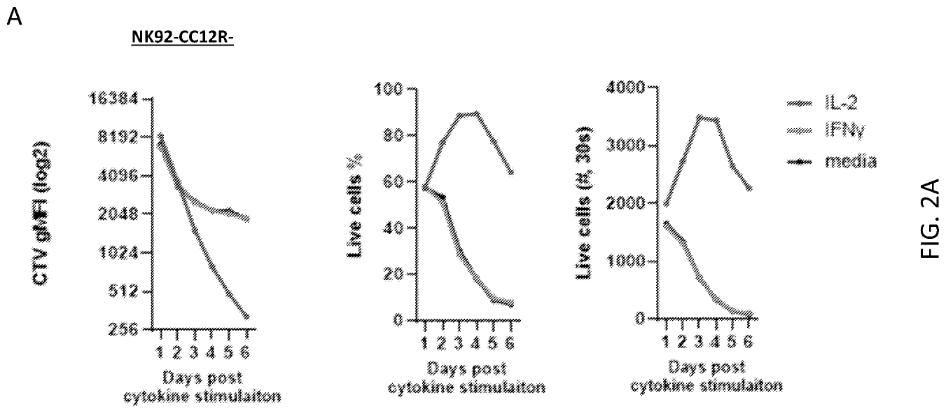

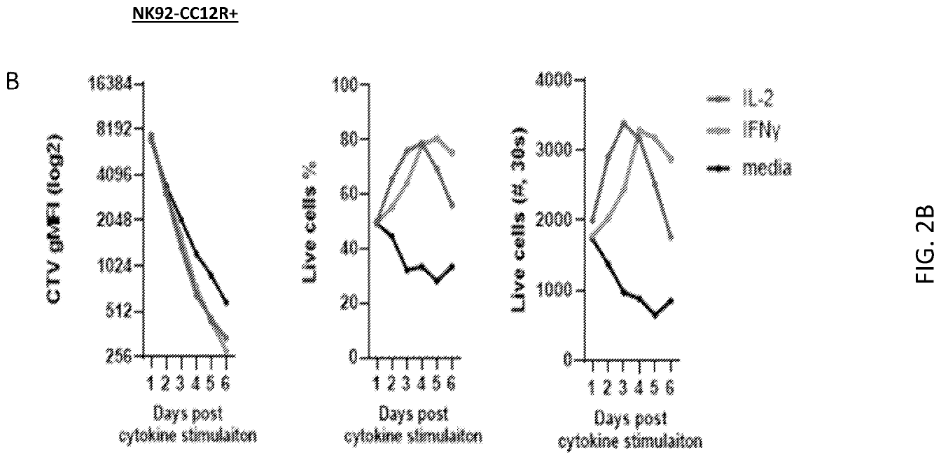

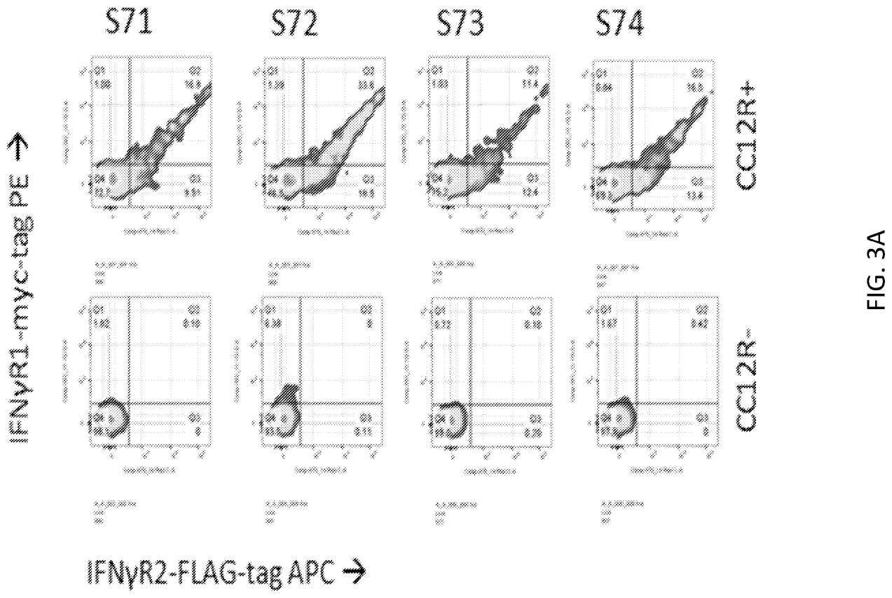

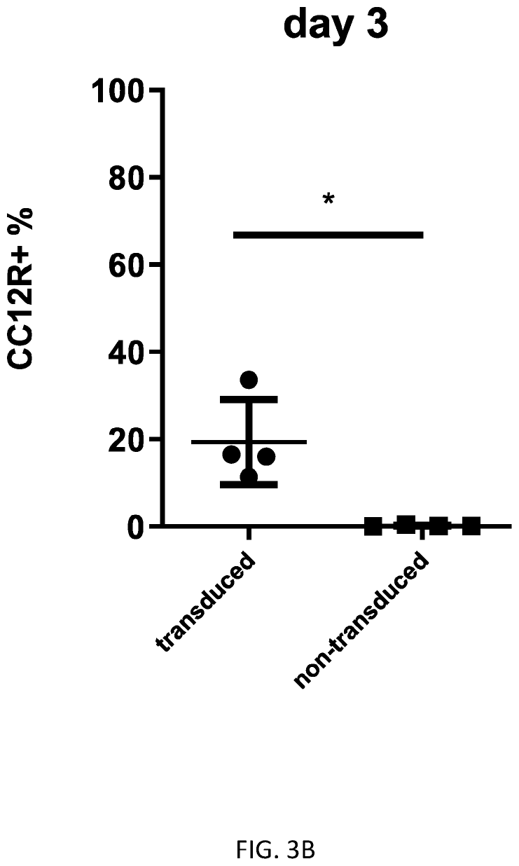

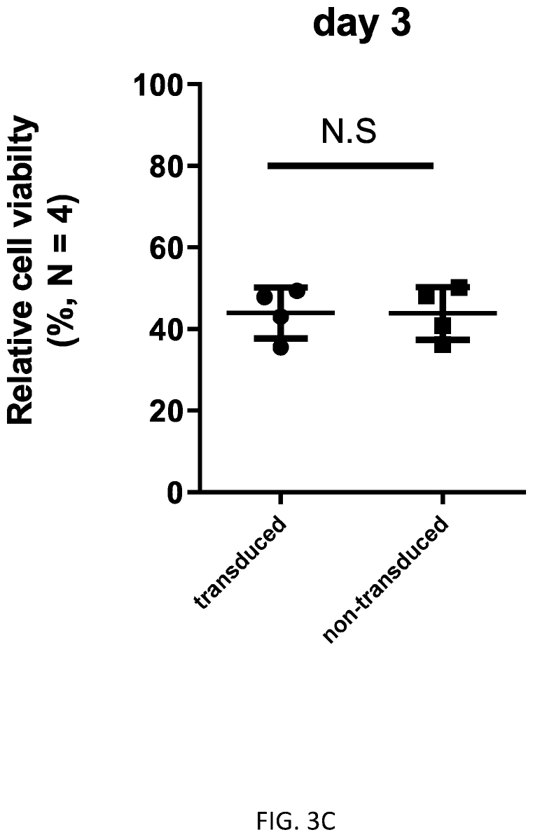

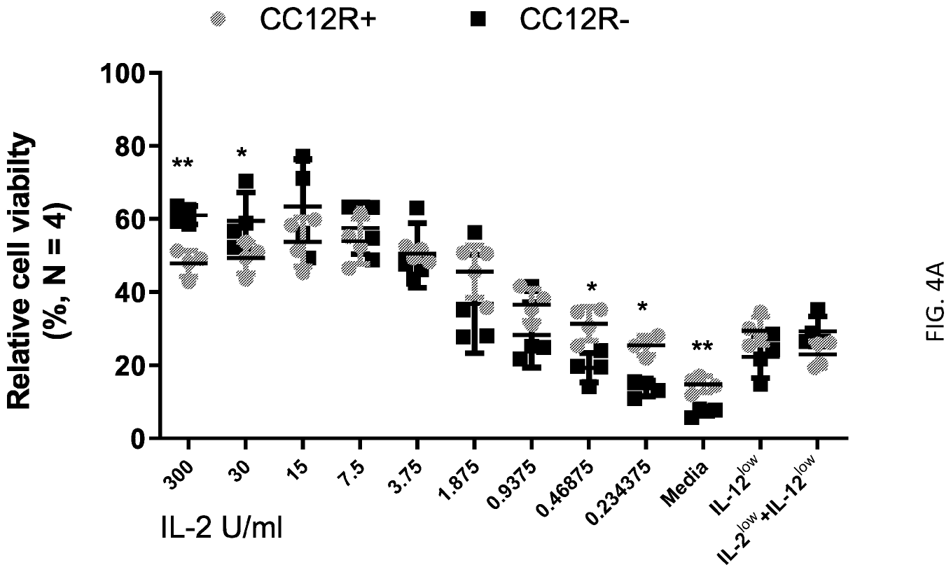

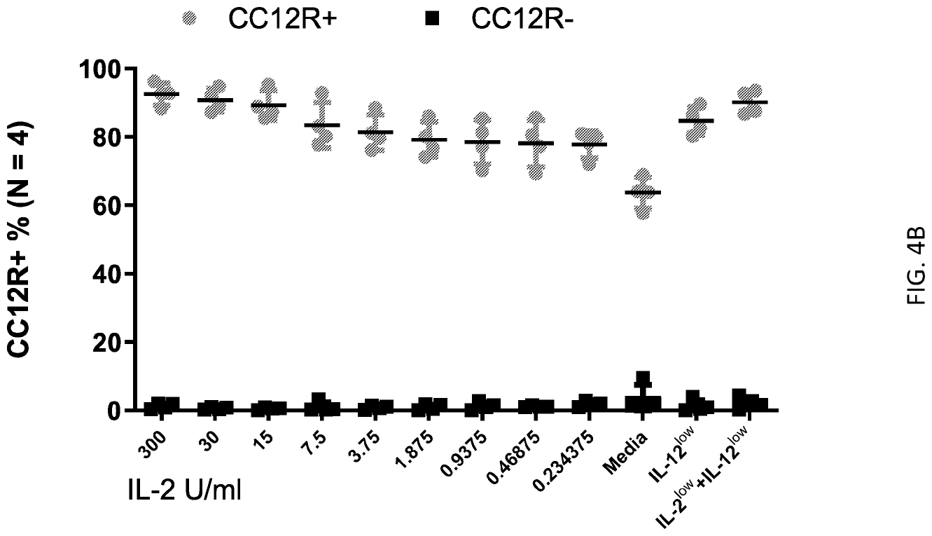

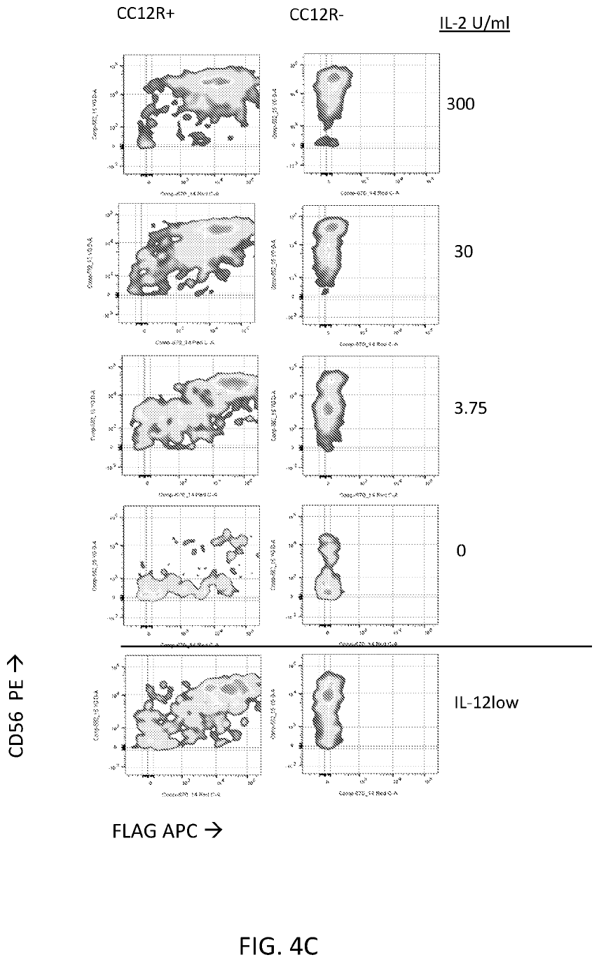

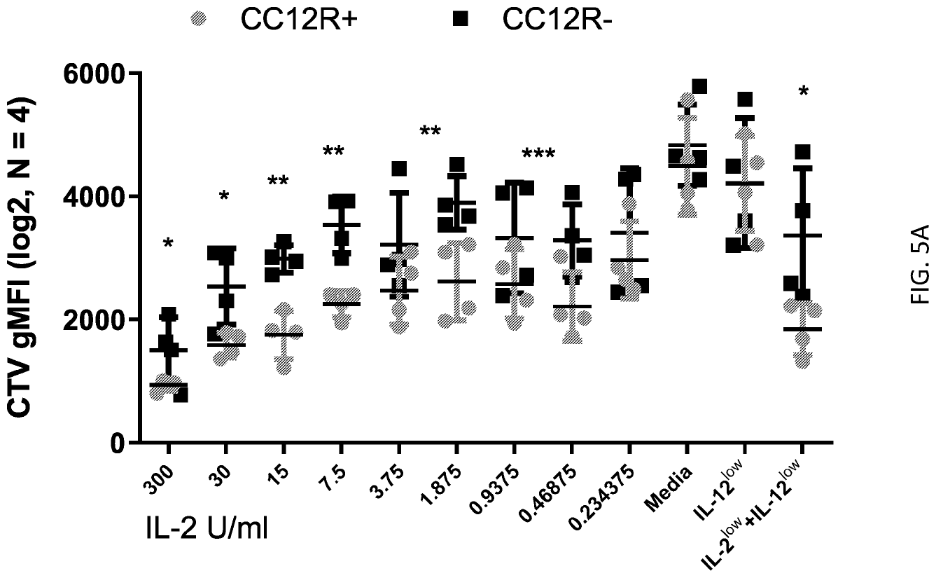

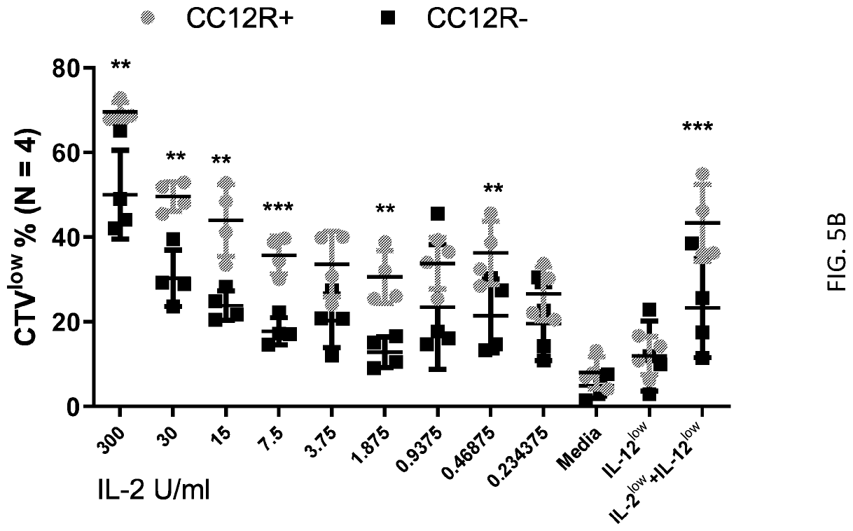

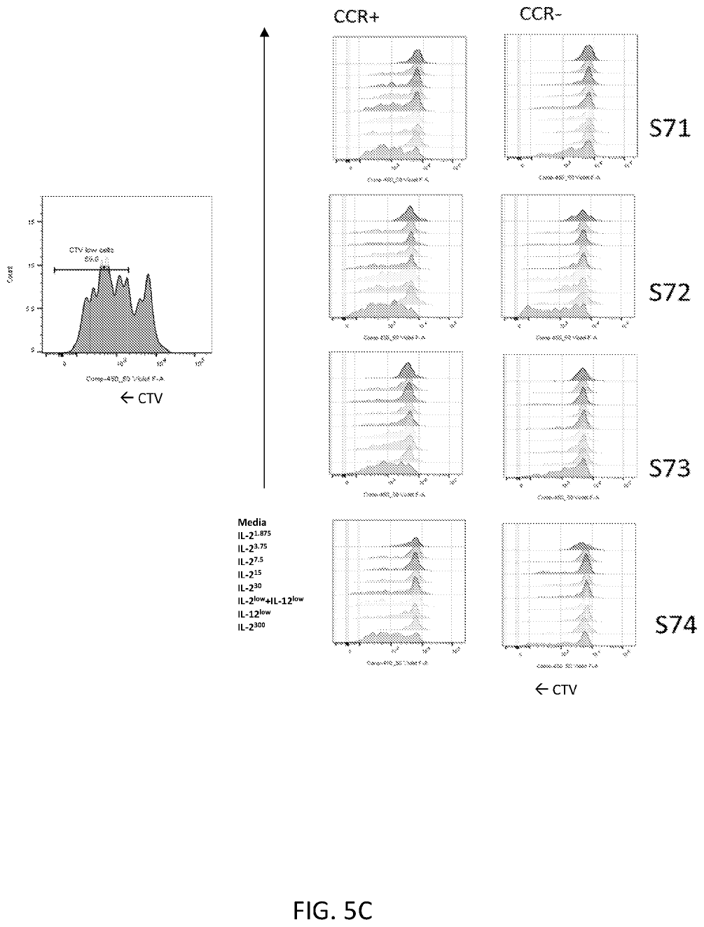

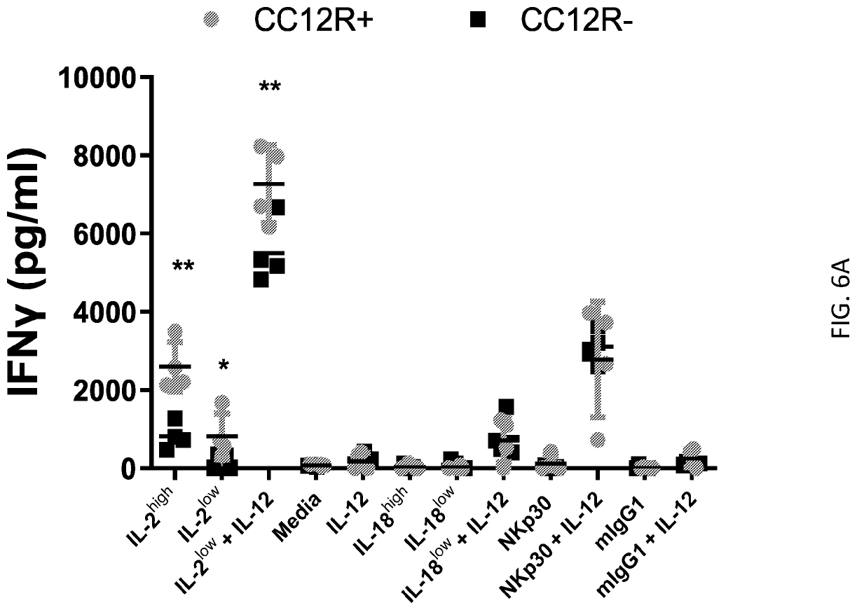

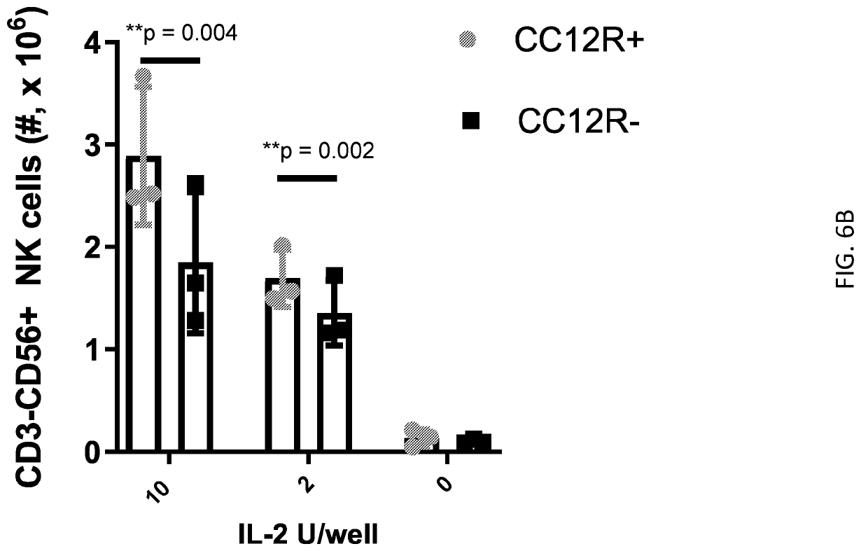

We designed a chimeric cytokine IL-12 receptor (CC12R) by fusion of the extracellular domains of the human IFNγ receptor (IFNγR1 and IFNγR2) with the transmembrane and intracellular domains of the human IL-12 receptor (IL12RB1 and IL12RB2) to avoid exogenous IL-12 stimulation (FIG. 1A). CC12R expression in the IL-2-dependent NK92 cell line sustained cell viability and proliferation by IFNγ stimulation without IL-2 or exogenous IL-12 (FIG. 2). Therefore, we transduced ex vivo primary NK cells with CC12R+ lentiviral particles (FIG. 3) and then assessed CC12R-mediated NK cell proliferation in the presence of increasing IL-2 concentrations. CC12R+-transduced primary NK cells exhibited a significant increase in NK cell proliferation relative to untransduced donor-matched CC12R− NK cells. The increase in NK cell proliferation was associated with increased IL-2 concentrations, indicating synergy with IL-2 signaling (FIG. 4, FIG. 5). Similarly, CC12R expression significantly increased IFNγ secretion relative to untransduced donor-matched CC12R− NK cells (FIG. 6A). However, CC12R expression promoted NK cell proliferation during co-culture with irradiated 721.221-membrane-bound human IL-21 target cells only during IL-2 co-stimulation (FIG. 6B) (Yang et al., 2020). Thus, we concluded that other factors associated with IL-2 or IL-15 priming, besides the upregulation of the IL-12 receptor chains, are necessary to promote IL-12-mediated proliferation. Methods

Human IFNγR-human IL12R or Human IFNγR-human IL2R chimeric cytokine receptor (CCR) constructs were cloned into the lentivirus vectors pHR containing the EF1a or SFFV promotor using an In-Fusion® HD Cloning Kit (TAKARA). The chimeric cytokine receptor contained amino acids 1-245 of IFNγR1 and amino acids 1-247 of IFNγR2 (extracellular domains of the human IFNγ receptor). CC12R transmembrane and intracellular domains are amino acids 546-662 of IL12RB1 and amino acids 663-862 of IL12RB2 of the human IL-12 receptor. CC2R transmembrane and intracellular domains are amino acids 241-551 of IL2RB and amino acids 263-369 of IL2RG of the IL-2/IL-15 receptor (Integrated DNA Technologies, IDT). Myc-tag was integrated into the N-terminus of the first CC12R or CC2R chain while FLAG-tag was integrated into the N-terminus of the second CC12R or CC2R chain to allow surface detection. CC12R or CC2R chains were separated by a T2A sequence. Lentivirus preparation was done by using the pMD2.G and pCMV dr8.91 packaging vectors and transfection of the Lenti-X™ 293T cell Line (TAKARA) cultured in complete DMEM plus 10% FCS. Lentivirus was concentrated using a Lenti-X™ concentrator (TAKARA) and resuspended in 1 ml RPMI-1640+10% fetal calf serum (FCS) with protamine sulfate (1 μg/ml). Aliquots were kept at −20° C.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A human natural killer cell or T-cell expressing a first and second chimeric transmembrane protein, wherein
the first chimeric transmembrane protein comprises a first ligand-binding ectodomain linked to one or more intracellular signaling domains from a human IL-12 receptor or IL-15 receptor; and
the second chimeric transmembrane protein comprises a second ligand-binding ectodomain linked to one or more intracellular signaling domains from a human IL-12 receptor or IL-15 receptor or other interleukin receptor,
wherein the first and second ligand-binding ectodomains together bind the ligand to trigger signaling by the intracellular signaling domains.

2. The human natural killer cell or T-cell of claim 1, wherein intracellular signaling domains of the first chimeric transmembrane protein and the second chimeric transmembrane protein are human IL-12 receptor signaling domains.

3. The human natural killer cell or T-cell of claim 2, wherein at least one of the one or more intracellular signaling domains in the first chimeric transmembrane receptor comprise a signaling domain from human IL12RB1, and
at least one of the one or more intracellular signaling domains in the second chimeric transmembrane receptor comprise a signaling domain from human IL12RB2.

4. The human natural killer cell or T-cell of claim 3, wherein the signaling domain from human IL12RB1 comprises an amino acid sequence at least 90% (e.g., 91, 92, 93, 94, 95, 96, 97, 98, 99, or 100%) identical to SEQ ID NO: 7; and
the signaling domain from human IL12RB2 comprises an amino acid sequence at least 90% (e.g., 91, 92, 93, 94, 95, 96, 97, 98, 99, or 100%) identical to SEQ ID NO: 8.

5. The human natural killer cell or T-cell of claim 1, wherein intracellular signaling domains of the first chimeric transmembrane protein and the second chimeric transmembrane protein are human IL-15 receptor signaling domains.

6. The human natural killer cell or T-cell of claim 5, wherein at least one of the one or more intracellular signaling domains in the first chimeric transmembrane receptor comprise a signaling domain from human IL2RB, and
at least one of the one or more intracellular signaling domains in the second chimeric transmembrane receptor comprise a signaling domain from human IL2RG.

7. The human natural killer cell or T-cell of claim 6, wherein the signaling domain from human IL2RB comprises an amino acid sequence at least 90% (e.g., 91, 92, 93, 94, 95, 96, 97, 98, 99, or 100%) identical to SEQ ID NO: 9; and
the signaling domain from human IL2RG comprises an amino acid sequence at least 90% (e.g., 91, 92, 93, 94, 95, 96, 97, 98, 99, or 100%) identical to SEQ ID NO: 10.

8. The human natural killer cell or T-cell of any one of claims 1-7, wherein the ligand is IFNγ and the first ligand-binding ectodomain comprises a human IFNγR1 IFNγ-binding domain and the second ligand-binding ectodomain comprises a human IFNγR2 IFNγ-binding domain.

9. The human natural killer cell or T-cell of any one of claims 1-7, wherein the ligand is IFNγ and the first ligand-binding ectodomain comprises a human IFNγR2 IFNγ-binding domain and the second ligand-binding ectodomain comprises a human IFNγR1 IFNγ-binding domain.

10. The human natural killer cell or T-cell of any one of claim 8 or 9, wherein the human IFNγR1 IFNγ-binding domain comprises an amino acid sequence at least 90% (e.g., 91, 92, 93, 94, 95, 96, 97, 98, 99, or 100%) identical to SEQ ID NO: 1; and
the human IFNγR2 IFNγ-binding domain comprises an amino acid sequence at least 90% (e.g., 91, 92, 93, 94, 95, 96, 97, 98, 99, or 100%) identical to SEQ ID NO: 2.

11. The human natural killer cell or T-cell of any one of claims 1-7, wherein the ligand is GM-CSF and the first ligand-binding ectodomain comprises a human CSF2RA GM-CSF binding domain and the second ligand-binding ectodomain comprises a human CSF2RB GM-CSF-binding domain.

12. The human natural killer cell or T-cell of any one of claims 1-7, wherein the ligand is GM-CSF and the first ligand-binding ectodomain comprises a human CSF2RB GM-CSF-binding domain and the second ligand-binding ectodomain comprises a human CSF2RA GM-CSF-binding domain.

13. The human natural killer cell or T-cell of any one of claim 11 or 12, wherein the human CSF2RA GM-CSF-binding domain comprises an amino acid sequence at least 90% (e.g., 91, 92, 93, 94, 95, 96, 97, 98, 99, or 100%) identical to SEQ ID NO: 3; and
the human CSF2RB GM-CSF-binding domain comprises an amino acid sequence at least 90% (e.g., 91, 92, 93, 94, 95, 96, 97, 98, 99, or 100%) identical to SEQ ID NO: 4.

14. The human natural killer cell or T-cell of any one of claims 1-7, wherein the ligand is TGFβ and the first ligand-binding ectodomain comprises a human TGFBR1 TGFβ-binding domain and the second ligand-binding ectodomain comprises a human TGFBR2 TGFβ-binding domain.

15. The human natural killer cell or T-cell of any one of claims 1-7, wherein the ligand is TGFβ and the first ligand-binding ectodomain comprises a human TGFBR2 TGFβ-binding domain and the second ligand-binding ectodomain comprises a human TGFBR1 TGFβ-binding domain.

16. The human natural killer cell or T-cell of any one of claim 8 or 9, wherein the human TGFBR1 TGFβ-binding domain comprises an amino acid sequence at least 90% (e.g., 91, 92, 93, 94, 95, 96, 97, 98, 99, or 100%) identical to SEQ ID NO: 5; and
the human TGFBR2 TGFβ-binding domain comprises an amino acid sequence at least 90% (e.g., 91, 92, 93, 94, 95, 96, 97, 98, 99, or 100%) identical to SEQ ID NO: 6.

17. The human natural killer cell or T-cell of claim 2, wherein at least one of the one or more intracellular signaling domains in the first chimeric transmembrane receptor comprise a signaling domain from human IL12RB1, and
at least one of the one or more intracellular signaling domains in the second chimeric transmembrane receptor comprise a signaling domain from human IL23R; or
wherein at least one of the one or more intracellular signaling domains in the first chimeric transmembrane receptor comprise a signaling domain from human IL12RB2, and
at least one of the one or more intracellular signaling domains in the second chimeric transmembrane receptor comprise a signaling domain from human GP130; or
wherein at least one of the one or more intracellular signaling domains in the first chimeric transmembrane receptor comprise a signaling domain from human IL12RB2, and
at least one of the one or more intracellular signaling domains in the second chimeric transmembrane receptor comprise a signaling domain from human IL27RA or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL21R or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL4R or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL7R or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL9R; or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL12RB1; or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL12RB2; or
wherein at least one of the one or more intracellular signaling domains in the first chimeric transmembrane receptor comprise a signaling domain from human IL2RB, and
at least one of the one or more intracellular signaling domains in the second chimeric transmembrane receptor comprise a signaling domain from human IL12RB1; or
wherein at least one of the one or more intracellular signaling domains in the first chimeric transmembrane receptor comprise a signaling domain from human IL2RB, and
at least one of the one or more intracellular signaling domains in the second chimeric transmembrane receptor comprise a signaling domain from human IL12RB2.

18. The human natural killer cell or T-cell of any one of claims 1-17, wherein the human natural killer cell is a primary natural killer cell or derived from an induced pluripotent stem cell.

19. A first nucleic acid and a second nucleic acid, the first nucleic acid encoding a first chimeric transmembrane protein that comprises a first ligand-binding ectodomain linked to one or more intracellular signaling domains from a human IL12 receptor or IL-15 receptor; and
the second nucleic acid encoding a second chimeric transmembrane protein that comprises a second ligand-binding ectodomain linked to one or more intracellular signaling domains from a human IL-12 receptor or IL-15 receptor,
wherein when the first chimeric transmembrane protein and the second chimeric transmembrane protein are expressed in a cell in the presence of the ligand, the first and second ligand-binding ectodomains together bind the ligand to trigger signaling by the intracellular signaling domains.

20. The first nucleic acid and a second nucleic acid of claim 19, wherein intracellular signaling domains of the first chimeric transmembrane protein and the second chimeric transmembrane protein are human IL-12 receptor signaling domains.

21. The first nucleic acid and a second nucleic acid of claim 20, wherein at least one of the one or more intracellular signaling domains in the first chimeric transmembrane receptor comprise a signaling domain from human IL12RB1, and
at least one of the one or more intracellular signaling domains in the second chimeric transmembrane receptor comprise a signaling domain from human IL12RB2.

22. The first nucleic acid and a second nucleic acid of claim 21, wherein the signaling domain from human IL12RB1 comprises an amino acid sequence at least 90% (e.g., 91, 92, 93, 94, 95, 96, 97, 98, 99, or 100%) identical to SEQ ID NO: 7; and
the signaling domain from human IL12RB2 comprises an amino acid sequence at least 90% (e.g., 91, 92, 93, 94, 95, 96, 97, 98, 99, or 100%) identical to SEQ ID NO: 8.

23. The first nucleic acid and a second nucleic acid of claim 19, wherein intracellular signaling domains of the first chimeric transmembrane protein and the second chimeric transmembrane protein are human IL-15 receptor signaling domains.

24. The first nucleic acid and a second nucleic acid of claim 23, wherein at least one of the one or more intracellular signaling domains in the first chimeric transmembrane receptor comprise a signaling domain from human IL2RB, and
at least one of the one or more intracellular signaling domains in the second chimeric transmembrane receptor comprise a signaling domain from human IL2RG.

25. The first nucleic acid and a second nucleic acid of claim 24, wherein the signaling domain from human IL2RB comprises an amino acid sequence at least 90% (e.g., 91, 92, 93, 94, 95, 96, 97, 98, 99, or 100%) identical to SEQ ID NO: 9; and
the signaling domain from human IL2RG comprises an amino acid sequence at least 90% (e.g., 91, 92, 93, 94, 95, 96, 97, 98, 99, or 100%) identical to SEQ ID NO: 10.

26. The first nucleic acid and a second nucleic acid of any one of claims 19-25, wherein the ligand is IFNγ and the first ligand-binding ectodomain comprises a human IFNγR1 IFNγ-binding domain and the second ligand-binding ectodomain comprises a human IFNγR2 IFNγ-binding domain.

27. The first nucleic acid and a second nucleic acid of any one of claims 19-25, wherein the ligand is IFNγ and the first ligand-binding ectodomain comprises a human IFNγR2 IFNγ-binding domain and the second ligand-binding ectodomain comprises a human IFNγR1 IFNγ-binding domain.

28. The first nucleic acid and a second nucleic acid of any one of claim 26 or 27, wherein the human IFNγR1 IFNγ-binding domain comprises an amino acid sequence at least 90% (e.g., 91, 92, 93, 94, 95, 96, 97, 98, 99, or 100%) identical to SEQ ID NO: 1 and
the human IFNγR2 IFNγ-binding domain comprises an amino acid sequence at least 90% (e.g., 91, 92, 93, 94, 95, 96, 97, 98, 99, or 100%) identical to SEQ ID NO: 2.

29. The first nucleic acid and the second nucleic acid of any one of claims 19-25, wherein the ligand is GM-CSF and the first ligand-binding ectodomain comprises a human CSF2RA GM-CSF binding domain and the second ligand-binding ectodomain comprises a human CSF2RB GM-CSF-binding domain.

30. The first nucleic acid and the second nucleic acid of any one of claims 19-25, wherein the ligand is GM-CSF and the first ligand-binding ectodomain comprises a human CSF2RB GM-CSF-binding domain and the second ligand-binding ectodomain comprises a human CSF2RA GM-CSF-binding domain.

31. The first nucleic acid and the second nucleic acid of any one of claim 30 or 31, wherein the human CSF2RA GM-CSF-binding domain comprises an amino acid sequence at least 90% (e.g., 91, 92, 93, 94, 95, 96, 97, 98, 99, or 100%) identical to SEQ ID NO: 3; and
the human CSF2RB GM-CSF-binding domain comprises an amino acid sequence at least 90% (e.g., 91, 92, 93, 94, 95, 96, 97, 98, 99, or 100%) identical to SEQ ID NO: 4.

32. The first nucleic acid and the second nucleic acid of any one of claims 19-25, wherein the ligand is TGFβ and the first ligand-binding ectodomain comprises a human TGFBR1 TGFβ-binding domain and the second ligand-binding ectodomain comprises a human TGFBR2 TGFβ-binding domain.

33. The first nucleic acid and the second nucleic acid of any one of claims 19-25, wherein the ligand is TGFβ and the first ligand-binding ectodomain comprises a human TGFBR2 TGFβ-binding domain and the second ligand-binding ectodomain comprises a human TGFBR1 TGFβ-binding domain.

34. The first nucleic acid and the second nucleic acid of any one of claim 32 or 33, wherein the human TGFBR1 TGFβ-binding domain comprises an amino acid sequence at least 90% (e.g., 91, 92, 93, 94, 95, 96, 97, 98, 99, or 100%) identical to SEQ ID NO: 5; and
the human TGFBR2 TGFβ-binding domain comprises an amino acid sequence at least 90% (e.g., 91, 92, 93, 94, 95, 96, 97, 98, 99, or 100%) identical to SEQ ID NO: 6.

35. The first nucleic acid and a second nucleic acid of claim 20, wherein at least one of the one or more intracellular signaling domains in the first chimeric transmembrane receptor comprise a signaling domain from human IL12RB1, and
at least one of the one or more intracellular signaling domains in the second chimeric transmembrane receptor comprise a signaling domain from human IL23R; or
wherein at least one of the one or more intracellular signaling domains in the first chimeric transmembrane receptor comprise a signaling domain from human IL12RB2, and
at least one of the one or more intracellular signaling domains in the second chimeric transmembrane receptor comprise a signaling domain from human GP130; or
wherein at least one of the one or more intracellular signaling domains in the first chimeric transmembrane receptor comprise a signaling domain from human IL12RB2, and
at least one of the one or more intracellular signaling domains in the second chimeric transmembrane receptor comprise a signaling domain from human IL27RA or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL21R or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL4R or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL7R or
wherein at least one of the one or more intracellular signaling domains in the first chimeric transmembrane receptor comprise a signaling domain from human IL2RG, and
at least one of the one or more intracellular signaling domains in the second chimeric transmembrane receptor comprise a signaling domain from human IL9R.

36. The first nucleic acid and the second nucleic acid of any one of claims 19-35, wherein the first nucleic acid and the second nucleic acid are linked together as one polynucleotide.

37. The first nucleic acid and the second nucleic acid of claim 36, wherein the first nucleic acid and second nucleic acid comprise a single open reading frame that encodes the first chimeric transmembrane protein linked to the second chimeric transmembrane protein via a cleavable amino acid sequence.

38. The first nucleic acid and the second nucleic acid of claim 37, wherein the cleavable amino acid sequence comprises one or more of a T2a peptide sequence, a P2A peptide sequence, an E2A peptide sequence, an F2A peptide sequence or a furin-cleavable sequence.

39. The first nucleic acid and the second nucleic acid of any one of claims 19-35, wherein the first nucleic acid and the second nucleic acid are separate polynucleotides not linked together.

40. A vector comprising the one polynucleotide of claim 36.

41. The vector of claim 40, wherein the vector is a viral vector or a plasmid.

42. A cell comprising the first nucleic acid and the second nucleic acid of any one of claims 19-39 or comprising the vector of claim 40 or 41.

43. A method of making a human natural killer cell or T-cell expressing a first and second chimeric transmembrane protein, wherein
the first chimeric transmembrane protein comprises a first ligand-binding ectodomain linked to one or more intracellular signaling domains from a human IL-12 receptor or IL-15 receptor; and
the second chimeric transmembrane protein comprises a second ligand-binding ectodomain linked to one or more intracellular signaling domains from a human IL-12 receptor or IL-15 receptor, and
the first and second ligand-binding ectodomains together bind the ligand to trigger signaling by the intracellular signaling domains, the method comprising,
introducing the first nucleic acid and the second nucleic acid of any one of claims 19-39 into a human natural killer cell or T-cell under conditions to allow for expression of the first and second chimeric transmembrane protein.

44. The method of claim 43, wherein the natural killer cell is a primary natural killer cell.

45. The method of claim 43 or 44, following the introducing, administering the natural killer cells or T-cells to a human.

46. The method of claim 45, wherein the natural killer cells or T-cells are autologous or allogenic to the human.

47. A method of stimulating natural killer cell or T-cell proliferation, the method comprising,
contacting a ligand to natural killer cells or T-cells expressing the first and second chimeric transmembrane protein or any one of claims 1-18, wherein
the first and second ligand-binding ectodomains together bind the ligand to trigger signaling by the intracellular signaling domains and stimulates natural killer cell or T-cell proliferation.

48. The method of claim 47, wherein the contacting is performed in vitro.

49. The method of claim 47, wherein the contacting is performed in vivo or ex vivo.

50. The method of claim 47, wherein the natural killer cell or T-cell produce the ligand.


SEQUENCE LISTING (104 sequences)

SEQ ID NO: 1 - protein, 228 aa, synthetic construct [sequence omitted]

SEQ ID NO: 2 - protein, 245 aa, synthetic construct
  REGION 1..245: note = Description of sequence: Enzyme: Archetype sequence
APPAQLTLETYQEWCNDSAATHDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQ

SEQ ID NO: 3 - protein, 326 aa, synthetic construct
  REGION 1..326: note = Description of sequence: Enzyme: Archetype sequence
MLLLVTSLLLCELPHPAFLHHHHHHLIPEKSDLRTVAPASSLNVRFDSRTMNLSWDCQENTTFSKCFLTDKKNRVVEPRLSNNECSCTFREICLHEGVTFEVHVNTSQRGFQQKLLYPNSGREGTAAQNFSCFIYNADLMNCTWARGPTAPRDVQYFLYIRNSKRRREIRCPYYIQDSGTHVGCHLDNLSGLTSRNYFLVNGTSREIGIQFFDSLLDTKKIERFNPPSNVTVRCNTTHCLVRWKQPRTYQKLSYLDFQYQLDVHRKNTQPGTENLLINVSGDLENRYNFPSSEPRAKHSVKIRAADVRILNWSSWSEAIEFGSDDG

SEQ ID NO: 4 - protein, 427 aa, synthetic construct
  REGION 1..427: note = Description of sequence: Enzyme: Archetype sequence
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMW

SEQ ID NO: 5 - protein, 103 aa, synthetic construct
  REGION 1..103: note = Description of sequence: Enzyme: Archetype sequence
AAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVE

SEQ ID NO: 6 - protein, 169 aa, synthetic construct
  REGION 1..169: note = Description of sequence: Enzyme: Archetype sequence
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 7 - protein, 92 aa, synthetic construct
  REGION 1..92: note = Description of sequence: Enzyme: Archetype sequence
NRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 8 - protein, 219 aa, synthetic construct
  REGION 1..219: note = Description of sequence: Enzyme: Archetype sequence
HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 9 - protein, 286 aa, synthetic construct
  REGION 1..286: note = Description of sequence: Enzyme: Archetype sequence
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 10 - protein, 86 aa, synthetic construct
  REGION 1..86: note = Description of sequence: Enzyme: Archetype sequence
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 11 - protein, 253 aa, synthetic construct
  REGION 1..253: note = Description of sequence: Enzyme: Archetype sequence
NRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 12 - protein, 277 aa, synthetic construct
  REGION 1..277: note = Description of sequence: Enzyme: Archetype sequence
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 13 - protein, 99 aa, synthetic construct
  REGION 1..99: note = Description of sequence: Enzyme: Archetype sequence
TSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 14 - protein, 285 aa, synthetic construct
  REGION 1..285: note = Description of sequence: Enzyme: Archetype sequence
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 15 - protein, 480 aa, synthetic construct
  REGION 1..480: note = Description of sequence: Enzyme: Archetype sequence
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQT

SEQ ID NO: 16 - protein, 195 aa, synthetic construct
  REGION 1..195: note = Description of sequence: Enzyme: Archetype sequence
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 17 - protein, 230 aa, synthetic construct
  REGION 1..230: note = Description of sequence: Enzyme: Archetype sequence
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 18 - protein, 21 aa, synthetic construct
  REGION 1..21: note = Description of sequence: Enzyme: IFNGR1-transmembrane domain
SLWIPVVAALLLFLVLSLVFI

SEQ ID NO: 19 - protein, 21 aa, synthetic construct
  REGION 1..21: note = Description of sequence: Enzyme: IFNGR2-transmembrane domain
VILISVGTFSLLSVLAGACFF

SEQ ID NO: 20 - protein, 25 aa, synthetic construct
  REGION 1..25: note = Description of sequence: Enzyme: IL12R1 -transmembrane domain
WLIFFASLGSFLSILLVGVLGYLGL

SEQ ID NO: 21 - protein, 21 aa, synthetic construct
  REGION 1..21: note = Description of sequence: Enzyme: IL12R2-transmembrane domain
WMAFVAPSICIAIIMVGIFST

SEQ ID NO: 22 - protein, 25 aa, synthetic construct
  REGION 1..25: note = Description of sequence: Enzyme: IL2RB -transmembrane domain
IPWLGHLLVGLSGAFGFIILVYLLI

SEQ ID NO: 23 - protein, 21 aa, synthetic construct
  REGION 1..21: note = Description of sequence: Enzyme: IL2RG - transmembrane domain
VVISVGSMGLIISLLCVYFWL

SEQ ID NO: 24 - protein, 26 aa, synthetic construct
  REGION 1..26: note = Description of sequence: Enzyme: CSF2RA transmembrane domain
NLGSVYIYVLLIVGTLVCGIVLGFLF

SEQ ID NO: 25 - protein, 17 aa, synthetic construct
  REGION 1..17: note = Description of sequence: Enzyme: CSF2RB transmembrane domain
VLALIVIFLTIAVLLAL

SEQ ID NO: 26 - protein, 21 aa, synthetic construct
  REGION 1..21: note = Description of sequence: Enzyme: IL23R transmembrane domain
LLLGMIVFAVMLSILSLIGIF

SEQ ID NO: 27 - protein, 21 aa, synthetic construct
  REGION 1..21: note = Description of sequence: Enzyme: IL27R transmembrane domain
VLPGILFLWGLFLLGCGLSLA

SEQ ID NO: 28 - protein, 22 aa, synthetic construct
  REGION 1..22: note = Description of sequence: Enzyme: GP130 transmembrane domain
AIVVPVCLAFLLTTLLGVLFCF

SEQ ID NO: 29 - protein, 21 aa, synthetic construct
  REGION 1..21: note = Description of sequence: Enzyme: IL21R transmembrane domain
GWNPHLLLLLLLVIVFIPAFW

SEQ ID NO: 30 - protein, 24 aa, synthetic construct
  REGION 1..24: note = Description of sequence: Enzyme: IL4R transmembrane domain
LLLGVSVSCIVILAVCLLCYVSIT

SEQ ID NO: 31 - protein, 25 aa, synthetic construct
  REGION 1..25: note = Description of sequence: Enzyme: IL7R transmembrane domain
PILLTISILSFFSVALLVILACVLW

SEQ ID NO: 32 - protein, 21 aa, synthetic construct
  REGION 1..21: note = Description of sequence: Enzyme: IL9R transmembrane domain
GNTLVAVSIFLLLTGPTYLLF

SEQ ID NO: 33 - protein, 17 aa, synthetic construct
  REGION 1..17: note = Description of sequence: Enzyme: IFNGR1 signal peptide
MALLFLLPLVMQGVSRA

SEQ ID NO: 34 - protein, 8 aa, synthetic construct
  REGION 1..8: note = Description of sequence: Enzyme: FLAG
DYKDDDDK

SEQ ID NO: 35 - protein, 10 aa, synthetic construct
  REGION 1..10: note = Description of sequence: Enzyme: influenza virus haemagglutinin HA and c-Myc tags
EQKLISEEDL

SEQ ID NO: 36 - protein, 18 aa, synthetic construct
  REGION 1..18: note = Description of sequence: Enzyme: a T2a peptide sequence
EGRGSLLTCGDVEENPGP

SEQ ID NO: 37 - protein, 19 aa, synthetic construct
  REGION 1..19: note = Description of sequence: Enzyme: a P2A peptide
ATNFSLLKQAGDVEENPGP

SEQ ID NO: 38 - protein, 20 aa, synthetic construct
  REGION 1..20: note = Description of sequence: Enzyme: an E2A peptide sequence
QCTNYALLKLAGDVESNPGP

SEQ ID NO: 39 - protein, 22 aa, synthetic construct
  REGION 1..22: note = Description of sequence: Enzyme: an F2A peptide sequence
VKQTLNFDLLKLAGDVESNPGP

SEQ ID NO: 40 - protein, 904 aa, synthetic construct
  REGION 1..904: note = Description of sequence: Enzyme: IFNGR1-IL12RB1-IFNGR2-IL12RB2
MALLFLLPLVMQGVSRAEQKLISEEDLEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFINRAARHLCPPLPTPCASSAIEFPGGKETWQWINPVDFQEEASLQEALVVEMSWDKGERTEPLEKTELPEGAPELALDTELSLEDGDRCKAKMRKRREGRGSLLTCGDVEENPGPMRPTLLWSLLLLLGVFAAAAADYKDDDDKAPPAQLTLETYQEWCNDSAATHDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 41 - protein, 904 aa, synthetic construct
  REGION 1..904: note = Description of sequence: Enzyme: IFNGR1-IL12RB2-IFNGR2-IL12RB1
MALLFLLPLVMQGVSRAEQKLISEEDLEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFIHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLMLRKRREGRGSLLTCGDVEENPGPMRPTLLWSLLLLLGVFAAAAADYKDDDDKAPPAQLTLETYQEWCNDSAATHDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 42 - protein, 908 aa, synthetic construct
  REGION 1..908: note = Description of sequence: Enzyme: IFNGR1-IL12RB1-IFNGR2-IL12RB2
MALLFLLPLVMQGVSRAEQKLISEEDLEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKMRKRREGRGSLLTCGDVEENPGPMRPTLLWSLLLLLGVFAAAAADYKDDDDKAPPAQLTLETYQEWCNDSAATHDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 43 - protein, 908 aa, synthetic construct
  REGION 1..908: note = Description of sequence: Enzyme: IFNGR1-IL12RB2-IFNGR2-IL12RB1
MALLFLLPLVMQGVSRAEQKLISEEDLEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLMLRKRREGRGSLLTCGDVEENPGPMRPTLLWSLLLLLGVFAAAAADYKDDDDKAPPAQLTLETYQEWCNDSAATHDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 44 - protein, 969 aa, synthetic construct
  REGION 1..969: note = Description of sequence: Enzyme: IFNGR1-IL2RB-IFNGR2-IL2RG
MALLFLLPLVMQGVSRAEQKLISEEDLEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVRKRREGRGSLLTCGDVEENPGPMRPTLLWSLLLLLGVFAAAAADYKDDDDKAPPAQLTLETYQEWCNDSAATHDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 45 - protein, 17 aa, synthetic construct
  REGION 1..17: note = Description of sequence: Enzyme: IFNGR1 signal peptide
MALLFLLPLVMQGVSRA

SEQ ID NO: 46 - protein, 10 aa, synthetic construct
  REGION 1..10: note = Description of sequence: Enzyme: Myc-tag
EQKLISEEDL

SEQ ID NO: 47 - protein, 228 aa, synthetic construct [sequence omitted]

SEQ ID NO: 48 - protein, 21 aa, synthetic construct
  REGION 1..21: note = Description of sequence: Enzyme: IFNGR1-transmembrane domain
SLWIPVVAALLLFLVLSLVFI

SEQ ID NO: 49 - protein, 92 aa, synthetic construct; identical to SEQ ID NO: 7
  REGION 1..92: note = Description of sequence: Enzyme: IL12RB1- intracellular domain

SEQ ID NO: 50 - protein, 4 aa, synthetic construct
  REGION 1..4: note = Description of sequence: Enzyme: Furin
RKRR

SEQ ID NO: 51 - protein, 18 aa, synthetic construct
  REGION 1..18: note = Description of sequence: Enzyme: T2A) or E2A F2A P2A
EGRGSLLTCGDVEENPGP

SEQ ID NO: 52 - protein, 21 aa, synthetic construct
  REGION 1..21: note = Description of sequence: Enzyme: IFNGR2 signal peptide
MRPTLLWSLLLLLGVFAAAAA

SEQ ID NO: 53 - protein, 8 aa, synthetic construct
  REGION 1..8: note = Description of sequence: Enzyme: FLAG-tag
DYKDDDDK

SEQ ID NO: 54 - protein, 245 aa, synthetic construct; identical to SEQ ID NO: 2
  REGION 1..245: note = Description of sequence: Enzyme: IFNGR2-ectodomain

SEQ ID NO: 55 - protein, 21 aa, synthetic construct
  REGION 1..21: note = Description of sequence: Enzyme: IFNGR2-transmembrane domain
VILISVGTFSLLSVLAGACFF

SEQ ID NO: 56 - protein, 219 aa, synthetic construct; identical to SEQ ID NO: 8
  REGION 1..219: note = Description of sequence: Enzyme: IL12RB2- intracellular domain

SEQ ID NO: 57 - protein, 17 aa, synthetic construct
  REGION 1..17: note = Description of sequence: Enzyme: IFNGR1 signal peptide
MALLFLLPLVMQGVSRA

SEQ ID NO: 58 - protein, 10 aa, synthetic construct
  REGION 1..10: note = Description of sequence: Enzyme: Myc-tag
EQKLISEEDL

SEQ ID NO: 59 - protein, 228 aa, synthetic construct [sequence omitted]

SEQ ID NO: 60 - protein, 21 aa, synthetic construct
  REGION 1..21: note = Description of sequence: Enzyme: IFNGR1-transmembrane domain
SLWIPVVAALLLFLVLSLVFI

SEQ ID NO: 61 - protein, 4 aa, synthetic construct
  REGION 1..4: note = Description of sequence: Enzyme: Furin
RKRR

SEQ ID NO: 62 - protein, 219 aa, synthetic construct; identical to SEQ ID NO: 8
  REGION 1..219: note = Description of sequence: Enzyme: IL12RB2- intracellular domain

SEQ ID NO: 63 - protein, 18 aa, synthetic construct
  REGION 1..18: note = Description of sequence: Enzyme: T2A) or E2A F2A P2A
EGRGSLLTCGDVEENPGP

SEQ ID NO: 64 - protein, 21 aa, synthetic construct
  REGION 1..21: note = Description of sequence: Enzyme: IFNGR2 signal peptide
MRPTLLWSLLLLLGVFAAAAA

SEQ ID NO: 65 - protein, 8 aa, synthetic construct
  REGION 1..8: note = Description of sequence: Enzyme: FLAG-tag
DYKDDDDK

SEQ ID NO: 66 - protein, 245 aa, synthetic construct; identical to SEQ ID NO: 2
  REGION 1..245: note = Description of sequence: Enzyme: IFNGR2-ectodomain

SEQ ID NO: 67 - protein, 21 aa, synthetic construct
  REGION 1..21: note = Description of sequence: Enzyme: IFNGR2-transmembrane domain
VILISVGTFSLLSVLAGACFF

SEQ ID NO: 68 - protein, 92 aa, synthetic construct; identical to SEQ ID NO: 7
  REGION 1..92: note = Description of sequence: Enzyme: IL12RB1- intracellular domain

SEQ ID NO: 69 - protein, 17 aa, synthetic construct
  REGION 1..17: note = Description of sequence: Enzyme: IFNGR1 signal peptide
MALLFLLPLVMQGVSRA

SEQ ID NO: 70 - protein, 10 aa, synthetic construct
  REGION 1..10: note = Description of sequence: Enzyme: Myc-tag
EQKLISEEDL

SEQ ID NO: 71 - protein, 228 aa, synthetic construct [sequence omitted]

SEQ ID NO: 72 - protein, 25 aa, synthetic construct
  REGION 1..25: note = Description of sequence: Enzyme: IL12R1 -transmembrane domain
WLIFFASLGSFLSILLVGVLGYLGL

SEQ ID NO: 73 - protein, 92 aa, synthetic construct; identical to SEQ ID NO: 7
  REGION 1..92: note = Description of sequence: Enzyme: IL12RB1- intracellular domain

SEQ ID NO: 74 - protein, 4 aa, synthetic construct
  REGION 1..4: note = Description of sequence: Enzyme: Furin
RKRR

SEQ ID NO: 75 - protein, 18 aa, synthetic construct
  REGION 1..18: note = Description of sequence: Enzyme: T2A) or E2A F2A P2A
EGRGSLLTCGDVEENPGP

SEQ ID NO: 76 - protein, 21 aa, synthetic construct
  REGION 1..21: note = Description of sequence: Enzyme: IFNGR2 signal peptide
MRPTLLWSLLLLLGVFAAAAA

SEQ ID NO: 77 - protein, 8 aa, synthetic construct
  REGION 1..8: note = Description of sequence: Enzyme: FLAG-tag
DYKDDDDK

SEQ ID NO: 78 - protein, 245 aa, synthetic construct; identical to SEQ ID NO: 2
  REGION 1..245: note = Description of sequence: Enzyme: IFNGR2-ectodomain

SEQ ID NO: 79 - protein, 21 aa, synthetic construct
  REGION 1..21: note = Description of sequence: Enzyme: IL12R2-transmembrane domain
WMAFVAPSICIAIIMVGIFST

SEQ ID NO: 80 - protein, 219 aa, synthetic construct; identical to SEQ ID NO: 8
  REGION 1..219: note = Description of sequence: Enzyme: IL12RB2- intracellular domain

SEQ ID NO: 81 - protein, 17 aa, synthetic construct
  REGION 1..17: note = Description of sequence: Enzyme: IFNGR1 signal peptide
MALLFLLPLVMQGVSRA

SEQ ID NO: 82 - protein, 10 aa, synthetic construct
  REGION 1..10: note = Description of sequence: Enzyme: Myc-tag
EQKLISEEDL

SEQ ID NO: 83 - protein, 228 aa, synthetic construct [sequence omitted]

SEQ ID NO: 84 - protein, 21 aa, synthetic construct
  REGION 1..21: note = Description of sequence: Enzyme: IL12RB2-transmembrane domain
WMAFVAPSICIAIIMVGIFST

SEQ ID NO: 85 - protein, 219 aa, synthetic construct; identical to SEQ ID NO: 8
  REGION 1..219: note = Description of sequence: Enzyme: IL12RB2- intracellular domain

SEQ ID NO: 86 - protein, 4 aa, synthetic construct
  REGION 1..4: note = Description of sequence: Enzyme: Furin
RKRR

SEQ ID NO: 87 - protein, 18 aa, synthetic construct
  REGION 1..18: note = Description of sequence: Enzyme: T2A) or E2A F2A P2A
EGRGSLLTCGDVEENPGP

SEQ ID NO: 88 - protein, 21 aa, synthetic construct
  REGION 1..21: note = Description of sequence: Enzyme: IFNGR2 signal peptide
MRPTLLWSLLLLLGVFAAAAA

SEQ ID NO: 89 - protein, 8 aa, synthetic construct
  REGION 1..8: note = Description of sequence: Enzyme: FLAG-tag
DYKDDDDK

SEQ ID NO: 90 - protein, 245 aa, synthetic construct; identical to SEQ ID NO: 2
  REGION 1..245: note = Description of sequence: Enzyme: IFNGR2-ectodomain

SEQ ID NO: 91 - protein, 25 aa, synthetic construct
  REGION 1..25: note = Description of sequence: Enzyme: IL12RB1 -transmembrane domain
WLIFFASLGSFLSILLVGVLGYLGL

SEQ ID NO: 92 - protein, 92 aa, synthetic construct; identical to SEQ ID NO: 7
  REGION 1..92: note = Description of sequence: Enzyme: IL12RB1- intracellular domain

SEQ ID NO: 93 - protein, 17 aa, synthetic construct
  REGION 1..17: note = Description of sequence: Enzyme: IFNGR1 signal peptide
MALLFLLPLVMQGVSRA

SEQ ID NO: 94 - protein, 10 aa, synthetic construct
  REGION 1..10: note = Description of sequence: Enzyme: Myc-tag
EQKLISEEDL

SEQ ID NO: 95 - protein, 228 aa, synthetic construct [sequence omitted]

SEQ ID NO: 96 - protein, 25 aa, synthetic construct
  REGION 1..25: note = Description of sequence: Enzyme: IL2RB -transmembrane domain
IPWLGHLLVGLSGAFGFIILVYLLI

SEQ ID NO: 97 - protein, 286 aa, synthetic construct; identical to SEQ ID NO: 9
  REGION 1..286: note = Description of sequence: Enzyme: IL2RB- intracellular domain

SEQ ID NO: 98 - protein, 4 aa, synthetic construct
  REGION 1..4: note = Description of sequence: Enzyme: Furin
RKRR

SEQ ID NO: 99 - protein, 18 aa, synthetic construct
  REGION 1..18: note = Description of sequence: Enzyme: T2A) or E2A F2A P2A
EGRGSLLTCGDVEENPGP

SEQ ID NO: 100 - protein, 21 aa, synthetic construct
  REGION 1..21: note = Description of sequence: Enzyme: IFNGR2 signal peptide
MRPTLLWSLLLLLGVFAAAAA

SEQ ID NO: 101 - protein, 8 aa, synthetic construct
  REGION 1..8: note = Description of sequence: Enzyme: FLAG-tag
DYKDDDDK

SEQ ID NO: 102 - protein, 245 aa, synthetic construct; identical to SEQ ID NO: 2
  REGION 1..245: note = Description of sequence: Enzyme: IFNGR2-ectodomain

SEQ ID NO: 103 - protein, 21 aa, synthetic construct
  REGION 1..21: note = Description of sequence: Enzyme: IL2RG - transmembrane domain
VVISVGSMGLIISLLCVYFWL

SEQ ID NO: 104 - protein, 86 aa, synthetic construct; identical to SEQ ID NO: 10
  REGION 1..86: note = Description of sequence: Enzyme: IL2RG - intracellular domain